(19) United States
(12) Patent Application Publication
(10) Pub. No.: US 2018/0230160 A1
(43) Pub. Date: Aug. 16, 2018

(54) 2-AMINO-7A-PHENYL-3,4,4A,5,7,7A-HEXAHYDROFURO[3,4-B]PYRIDINES AS BACE1 INHIBITORS

(71) Applicant: H. Lundbeck A/S, Valby (DK)

(72) Inventors: Karsten Juhl, Greve (DK); Lena Tagmose, Lyngby (DK); Mauro Marigo, Skovlunde (DK); Ask Puschl, Frederiksberg (DK)

(21) Appl. No.: 15/751,777

(22) PCT Filed: Aug. 10, 2016
(86) PCT No.: PCT/EP2016/069043

§ 371 Date: Feb. 9, 2018

(30) Foreign Application Priority Data
Aug. 12, 2015 (DK) PA 2015 00462
Aug. 12, 2015 (DK) PA 2015 00466

(51) Int. Cl. C07D 491/04; A61P 25/28

(52) U.S. Cl. CPC C07D 491/04; A61P 25/28

(57) ABSTRACT

Compounds of the Formula (I) are provided which compounds inhibitors of BACE1.

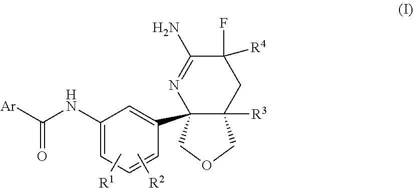

### FIELD OF THE INVENTION

The present invention provides compounds which are BACE1 inhibitors. Separate aspects of the invention are directed to pharmaceutical compositions comprising said compounds and uses of the compounds to treat neurodegenerative and cognitive disorders.

### BACKGROUND

Dementia is a clinical syndrome characterized by deficits in multiple areas of cognition that cannot be explained by normal aging, a noticeable decline in function, and an absence of delirium. In addition, neuropsychiatric symptoms and focal neurological findings are usually present. Dementia is further classified based on etiology. Alzheimer's disease (AD) is the most common cause of dementia, followed by mixed AD and vascular dementia, Lewy body dementia (DLB), and fronto-temporal dementia.

β-Amyloid deposits and neurofibrillary tangles are considered to be major pathologic characterizations associated with AD which is characterized by the loss of memory, cognition, reasoning, judgment, and orientation. Also affected, as the disease progresses, are motor, sensory and linguistic abilities until global impairment of multiple cognitive functions occurs. β-Amyloid deposits are predominantly an aggregate of Aβ peptide, which in turn is a product of the proteolysis of amyloid precursor protein (APP) as part of the β-amyloidogenic pathway. Aβ peptide results from the cleavage of APP at the C-terminals by one or more γ-secretases and at the N-terminus by β-secretase 1 (BACE1) also known as aspartyl protease 2. BACE1 activity is correlated directly to the generation of Aβ peptide from APP.

Studies indicate that the inhibition of BACE1 impedes the production of Aβ peptide. Further, BACE1 co-localizes with its substrate APP in Golgi and endocytic compartments (Willem M, et al. Semin. Cell Dev. Biol, 2009, 20, 175-182). Knock-out studies in mice have demonstrated the absence of amyloid peptide formation while the animals are healthy and fertile (Ohno M, et al. Neurobiol. Dis., 2007, 26, 134-145). Genetic ablation of BACE1 in APP-overexpressing mice has demonstrated absence of plaque formation, and the reverse of cognitive deficits (Ohno M, et al. Neuron; 2004, 41, 27-33). BACE1 levels are elevated in the brains of sporadic AD patients (Hampel and Shen, Scand. J. Clin. Lab. Invest. 2009, 69, 8-12).

These convergent findings indicate that the inhibition of BACE1 may be a therapeutic target for the treatment of AD as well as neurodegenerative or cognitive disorders for which the reduction of Aβ deposits is beneficial.

AstraZeneca announced the discovery of AZD3839, a potent BACE1 inhibitor clinical candidate for the treatment of AD (Jeppsson, F., et al. J. Biol. Chem., 2012, 287, 41245-41257) in October 2012. The effort which led to the discovery of AZD3839 was further described in Ginman, T., et al. J. Med. Chem., 2013, 56, 4181-4205. The Ginman publication describes the issues which were overcome in connection with the discovery and identification of AZD3839. These issues related to poor blood brain barrier penetration and P-glycoprotein mediated efflux of the compounds resulting in lack of brain exposure.

The Ginman manuscript hypothesized that the differences in brain exposure would largely be due to the core structures and Structure Activity Relationship data was provided wherein the in vitro properties on the reported compounds were given in four tables according to core sub-types. In table 4, a series of amidine containing compounds are described that were considered interesting from an activity perspective. However, the data suggests that the amidine containing core did not exhibit a favourable blood brain barrier permeability profile.

Researchers from Hoffmann-La Roche and Siena Biotech also reported the discovery of amidine containing compounds (Woltering, T. J., et al. Bioorg. Med. Chem. Lett. 2013, 23, 4239-4243). These compounds (compounds 17 and 18 in the paper) were found not to have any in vivo effect (lack of Aβ40 reduction in brain in wild type mice).

Contrary to the teachings of Ginman, et al. and Woltering, T. J., et al., the present inventors have discovered a series of amidine compounds which are brain penetrating. Accordingly, the present invention relates to novel compounds having BACE1 inhibitory activity, to their preparation, to their medical use and to medicaments comprising them.

### SUMMARY OF THE INVENTION

An objective of the present invention is to provide compounds that inhibit BACE1. Accordingly, the present invention relates to compounds of Formula I.

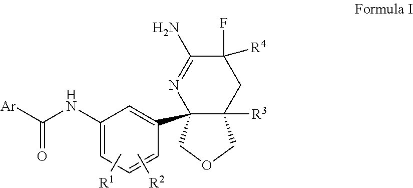

wherein Ar is selected from the group consisting of phenyl, pyridyl, pyrimidyl, pyrazinyl, imidazolyl, pyrazolyl, thiazolyl, oxazoly and isoxazolyl, and wherein Ar is optionally substituted with one or more substituents selected from the group consisting of halogen, CN, C₁-C₆ alkyl, C₂-C₆ alkenyl, C₂-C₆ alkynyl, C₁-C₆ fluoroalkyl and C₁-C₆ alkoxy;

R¹ is selected from the group consisting of hydrogen, halogen, C₁-C₃ alkyl and C₁-C₃ fluoroalkyl;

R² is selected from the group consisting of hydrogen, halogen, C₁-C₃ alkyl and C₁-C₃ fluoroalkyl;

R³ is selected from hydrogen or halogen;

R⁴ is selected from C₁-C₃ alkyl or C₁-C₃ fluoroalkyl;
or a pharmaceutically acceptable salt thereof.

In one embodiment, the present invention provides a compound of Formula I or a pharmaceutically acceptable salt thereof for use in therapy.

The present invention further provides a pharmaceutical composition comprising a compound of Formula I or a pharmaceutically acceptable salt thereof and a pharmaceutically acceptable carrier.

In one embodiment, the invention provides the use of a compound of Formula I or a pharmaceutically acceptable salt thereof in the manufacture of a medicament for the treatment of neurodegenerative or cognitive disorder.

In one embodiment, the invention provides a compound of Formula I or a pharmaceutically acceptable salt thereof for use in a method for the treatment of a neurodegenerative or cognitive disorder.

The present invention provides a method of treating a neurodegenerative or cognitive disorder comprising administering a therapeutically effective amount of a compound of Formula I or a pharmaceutically acceptable salt thereof to a patient in need thereof.

### DETAILED DESCRIPTION OF THE INVENTION

In one embodiment the invention provides compounds of Formula Ia wherein R¹-R⁴ and Ar are as defined above.

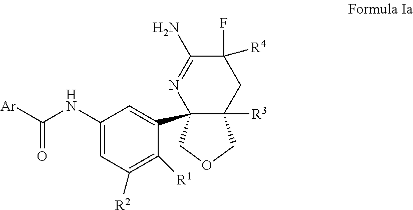

In one embodiment of the invention wherein the compound of the present invention is represented by Formula I or Formula Ia, R¹ is F, and in particular R¹ is F and R² is hydrogen.

In one embodiment of the present invention both R¹ and R² are F, in particular when the compounds of the present invention are represented by Formula Ia.

In one embodiment of the present invention R³ is selected from fluorine or hydrogen.

In one embodiment of the present invention R⁴ is selected from methyl or fluoromethyl.

In one embodiment of the invention, if R³ is hydrogen, then R⁴ is C₁-C₃ fluoroalkyl.

In one embodiment of the present invention, Ar is substituted with a substituent selected from F Cl, C₁-C₃ alkoxy or C₁-C₃ fluoroalkyl.

In one embodiment of the present invention, Ar is phenyl.

In one embodiment of the present invention, Ar is pyridyl.

In one embodiment of the present invention, Ar is pyrimidyl.

In one embodiment of the present invention, Ar is pyrazinyl.

In one embodiment of the present invention, Ar is imidazolyl.

In one embodiment of the present invention, Ar is pyrazolyl.

In one embodiment of the present invention, Ar is thiazolyl.

In one embodiment of the present invention, Ar is oxazolyl.

In one embodiment of the present invention, Ar is isoxazolyl.

In one embodiment of the present invention, a compound of the present invention is selected from the group consisting of N-(3-((3S,4aR,7aS)-2-amino-3,4a-difluoro-3-methyl-3,4,4a,5-tetrahydrofuro[3,4-b]pyridin-7a-yl)-4-fluorophenyl)-5-(methoxy-d₃)picolinamide,
N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-fluoropicolinamide,
N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-(difluoromethyl)pyrazine-2-carboxamide,
N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-methoxypicolinamide,
N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-methoxypyrazine-2-carboxamide,
N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-chloropicolinamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-methoxypyrazine-2-carboxamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-(difluoromethyl)pyrazine-2-carboxamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-4-methylthiazole-2-carboxamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-2-methyloxazole-4-carboxamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-methoxypicolinamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-fluoropicolinamide,
N-(3-((3R,4aR,7aS)-2-amino-3,4a-difluoro-3-methyl-3,4,4a,5-tetrahydrofuro[3,4-b]pyridin-7a-yl)-4-fluorophenyl)-5-(methoxy-d₃)pyrazine-2-carboxamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-chloropicolinamide,
N-(3-((3R,4aS,7aS)-2-amino-3-fluoro-3-(fluoromethyl)-3,4,4a,5-tetrahydrofuro[3,4-b]pyridin-7a-yl)-4-fluorophenyl)-5-methoxypicolinamide,
N-(3-((3R,4aS,7aS)-2-amino-3-fluoro-3-(fluoromethyl)-3,4,4a,5-tetrahydrofuro[3,4-b]pyridin-7a-yl)-4-fluorophenyl)-5-fluoropicolinamide,
N-[3-[(3R,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyridine-2-carboxamide,
N-[3-[(3R,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyrazine-2-carboxamide,
N-[3-[(3R,4aS,7aS)-2-amino-3-fluoro-3-(fluoromethyl)-4,4a,5,7-tetrahydrofuro[3,4-b]pyridin-7a-yl]-4,5-difluoro-phenyl]-5-fluoro-pyridine-2-carboxamide,
N-(3-((3S,4aS,7aS)-2-amino-3-fluoro-3-(fluoromethyl)-3,4,4a,5-tetrahydrofuro[3,4-b]pyridin-7a-yl)-4-fluorophenyl)-5-fluoropicolinamide,
N-(3-((3S,4aS,7aS)-2-amino-3-fluoro-3-(fluoromethyl)-3,4,4a,5,7,7a-hexahydrofuro[3,4-b]pyridin-7a-yl)-4-fluorophenyl)-5-methoxypicolinamide,
N-(3-((3S,4aS,7aS)-2-amino-3-fluoro-3-(fluoromethyl)-3,4,4a,5-tetrahydrofuro[3,4-b]pyridin-7a-yl)-4-fluorophenyl)-5-(methoxy-d₃)picolinamide,
N-[3-[(3S,4aS,7aS)-2-amino-3-fluoro-3-(fluoromethyl)-4,4a,5,7-tetrahydrofuro[3,4-b]pyridin-7a-yl]-4,5-difluoro-phenyl]-5-fluoro-pyridine-2-carboxamide,
N-[3-[(3S,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyrazine-2-carboxamide and
N-[3-[(3S,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyridine-2-carboxamide, or
   or a pharmaceutically acceptable salt thereof.

As used herein, the term “C₁-C₆ alkyl” refers to a straight chained or branched saturated hydrocarbon having from one to six carbon atoms inclusive. Examples of C₁-C₆ alkyl include, but are not limited to, methyl, ethyl, 1-propyl, 2-propyl, 1-butyl, 2-butyl, 2-methyl-2-propyl, 2-methyl-1-propyl, n-pentyl and n-hexyl. Similarly, the term “C₁-C₃ alkyl” refers to a straight chained or branched saturated hydrocarbon having from one to three carbon atoms inclusive. Examples of such substituents include, but are not limited to, methyl, ethyl and n-propyl.

Likewise, the term “C₁-C₆ alkoxy” refers to a straight chained or branched saturated alkoxy group having from one to six carbon atoms inclusive with the open valency on the oxygen. Examples of C₁-C₆ alkoxy include, but are not limited to, methoxy, ethoxy, n-butoxy, t-butoxy and n-hexyloxy. The “C₁-C₆ alkoxy” is optionally substituted with one or more fluorine atoms. As used herein, the term “C₁-C₆ fluoroalkyl” refers to a straight chained or branched saturated hydrocarbon having from one to six carbon atoms inclusive substituted with one or more fluorine atoms. Examples of C₁-C₆ fluoroalkyl include, but are not limited to, trifluoromethyl, pentafluoroethyl, 1-fluoroethyl, monofluoromethyl, difluoromethyl, 1,2-difluoroethyl and 3,4 difluorohexyl. Similarly, the term “C₁-C₃ fluoroalkyl” refers to a straight chained or branched saturated hydrocarbon having from one to three carbon atoms inclusive substituted with one or more fluorine atoms per carbon atom.

The term “halogen” refers to fluorine, chlorine, bromine and iodine.

The term “C₂-C₆ alkenyl” refers to a branched or unbranched alkenyl group having from two to six carbon atoms and one double bond, including but not limited to ethenyl, propenyl, and butenyl.

The term “C₂-C₆ alkynyl” shall mean a branched or unbranched alkynyl group having from two to six carbon atoms and one triple bond, including but not limited to ethynyl, propynyl and butynyl.

The phrase “therapeutically effective amount” when applied to a compound of the invention is intended to denote an amount of the compound that is sufficient to ameliorate, palliate, stabilize, reverse, slow or delay the progression of a disorder or disease state, or of a symptom of the disorder or disease. In an embodiment, the method of the present invention provides for administration of combinations of compounds. In such instances, the “therapeutically effective amount” is the amount of a compound of the present invention in the combination sufficient to cause the intended biological effect.

The term “treatment” or “treating” as used herein means ameliorating or reversing the progress or severity of a disease or disorder, or ameliorating or reversing one or more symptoms or side effects of such disease or disorder. “Treatment” or “treating”, as used herein, also means to inhibit or block, as in retard, arrest, restrain, impede or obstruct, the progress of a system, condition or state of a disease or disorder. For purposes of this invention, “treatment” or “treating” further means an approach for obtaining beneficial or desired clinical results, where “beneficial or desired clinical results” include, without limitation, alleviation of a symptom, diminishment of the extent of a disorder or disease, stabilized (i.e., not worsening) disease or disorder state, delay or slowing of a disease or disorder state, amelioration or palliation of a disease or disorder state, and remission of a disease or disorder, whether partial or total.

The present invention is based on the discovery that compounds of Formula I are inhibitors of BACE1, and as such, are useful for the treatment of disorders which pathological characteristics comprise 6-amyloid deposits and neurofibrillary tangles, such as neurodegenerative or cognitive disorders.

The compounds of the present invention are, as discussed above, expected to be useful in the treatment of Alzheimer's disease due to their effects on β-amyloid deposits and neurofibrillary tangles. This includes familial Alzheimer's disease where patients carry mutations on specific genes intimately involved in the production of Aβ peptide. It is, however, important to note that aggregates of Aβ peptide is not limited to familial Alzheimer's disease but is similarly an important pathophysiological characteristics of the more common sporadic Alzheimer's disease [Mol Cell Neurosci, 66, 3-11, 2015].

The compounds of the present invention are also believed to be useful in the treatment of early-stage Alzheimer's disease, i.e. disease stages where the biological and structural changes have started but the clinical manifestations of the disease have not yet become evident or are not yet well developed. Early-stage Alzheimer's disease may, in fact, start years before any clinical manifestation of the disease becomes manifest. Early-stage Alzheimer's disease includes prodromal Alzheimer's disease, preclinical Alzheimer's disease and mild cognitive impairment. Although mild cognitive impairment may be unrelated to Alzheimer's disease it is often a transitional stage to Alzheimer's disease or due to Alzheimer's disease. Preclinical and prodromal Alzheimer's disease are asymptomatic stages, and they are typically diagnosed by the presence of Alzheimer's disease related biomarkers. In this context the compounds of the present invention are believed to be useful in slowing down the progression of early-stage Alzheimer's disease, such as mild cognitive impairment to Alzheimer's disease. The compounds of the present invention are also believed to be useful in the treatment of memory loss, attention deficits and dementia associated with Alzheimer's disease.

Other diseases, in addition to the continuum of Alzheimer's disease, are characterized by β-amyloid deposits and neurofibrillary tangles. This includes e.g. Trisomy 21 also known as Down's syndrome. Patients suffering from Down's syndrome have an extra chromosome 21 which chromosome contains the gene for the amyloid precursor protein (APP). The extra chromosome 21 leads to overexpression of APP, which leads to increased levels of Aβ peptide, which eventually causes the markedly increased risk of developing Alzheimer's disease seen in Down's syndrome patients [Alzheimer's & Dementia, 11, 700-709, 201]. Cerebral amyloid angiopathy is also characterized by β-amyloid deposits and neurofibrillary tangles in blood vessels of the central nervous system [Pharmacol Reports, 67, 195-203, 2015] and is as such expected to be treatable with compounds of the present invention.

In one embodiment, the present invention provides a method of treating a disease selected from Alzheimer's disease (familial or sporadic), preclinical Alzheimer's disease, prodromal Alzheimer's disease, mild cognitive impairment, Down's syndrome and cerebral amyloid angiopathy, the method comprising the administration of a therapeutically effective amount of a compound of Formula I or a pharmaceutically acceptable salt thereof to a patient in need thereof.

The present invention further provides a method of inhibiting BACE1 in a patient comprising administering to a patient in need thereof a therapeutically effective amount of a compound of Formula I or a pharmaceutically acceptable salt thereof.

The present invention also provides a method of inhibiting 6-secretase mediated cleavage of amyloid precursor protein comprising administering to a patient in need of such treatment a therapeutically effective amount a compound of Formula I or a pharmaceutically acceptable salt thereof.

In further embodiments, the present invention provides the use of a compound of Formula I or a pharmaceutically acceptable salt thereof for the manufacture of a medicament for the treatment of disease selected from Alzheimer's disease (familial or sporadic), preclinical Alzheimer's disease, prodromal Alzheimer's disease, mild cognitive impairment, Down's syndrome or cerebral amyloid angiopathy.

The present invention also provides the use of a compound of Formula I or a pharmaceutically acceptable salt thereof for the manufacture of a medicament for the inhibition of BACE1. The present invention further provides the use of a compound of Formula I or a pharmaceutically acceptable salt thereof for the manufacture of a medicament for the inhibition of production or accumulation of Aβ peptide.

In one embodiment, the present invention provides a compound of Formula I or a pharmaceutically acceptable salt thereof for use in a method for the treatment of a disease selected form Alzheimer's disease (familial or sporadic), preclinical Alzheimer's disease, prodromal Alzheimer's disease, mild cognitive impairment, Down's syndrome or cerebral amyloid angiopathy.

In one embodiment, the present invention relates to a compound of Formula I or a pharmaceutically acceptable salt thereof for use in a method for inhibiting of BACE1 or in a method for inhibiting of production or accumulation of Aβ peptide.

The compounds of the present invention are as demonstrated in the Examples potent inhibitors of BACE1 and capable of lowering the level of Aβ peptide in rat brain and plasma, and said compounds are thus believed to be useful in the treatment of neurodegenerative and cognitive disorders which pathological characteristics comprise Aβ deposits and neurofibrilary tangles, such as e.g. Alzheimer's disease. It may be beneficial to combine a compound of the present invention with another treatment paradigm useful in the treatment of such disease, e.g. Alzheimer's disease.

Tau proteins are abundant in neurons. Tau proteins are soluble and highly phosphorylation labile and bind to tubulin providing regulation and modulation of tubulin assembly, i.e. eventually the microtubular structure and stability. Tau proteins can only associate with tubulin in the most de-phosphorylated state, and phosphorylation/de-phosphorylation acts as a switch controlling the tubulin association. Phosphorylated Tau constitutes an important part of the neurofibrillary tangles which are one of the hallmarks of Alzheimer's disease. The so-called Tau hypothesis suggests targeting these pathological tangles, a main constituent of which is phosphorylated Tau protein, as a treatment paradigm for Alzheimer's disease. In particular, immunotherapies, both active and passive, have been suggested as a way to target Tau neurofibrillary tangles. In active immunotherapy, a pathogenic antigen is injected into the patient and the innate immune system elicits an immune response. This triggers the maturation of B-cells generating high affinity antibodies against the administered antigen. In a passive immunotherapy, the triggering of the innate immune system is circumvented by infusing a specific antibody against the antigen. It is suggested that the inherent clearance system then removes antibody bound ligand. Substantial evidence for the efficacy of both active and passive immunotherapy targeting phosphorylated Tau protein as a treatment for Alzheimer's disease exists [Alzheimer's & Dementia, 7(4, suppl) S480-481; J Neurosci 30, 16559-16556, 2010; J Neurosci, 27, 9115-9129, 2007].

In one embodiment the invention provides a method for the treatment of a neurodegenerative or cognitive disorder, e.g. Alzheimer's disease, the method comprising the administration of a therapeutically effect amount of two components (1) a compound of Formula I or a pharmaceutically acceptable salt thereof and (2) a compound useful in active or passive Tau immunotherapy to a patient in need thereof. Said compound useful in passive Tau immunotherapy may be an antibody directed against phosphorylated Tau protein. Said compound useful in active Tau immunotherapy may be a fragment of the Tau protein amino acid sequence which upon injection in a patient elicits antibodies against phosphorylated Tau protein in said patient. The administration according to this embodiment of the invention may be simultaneous, or there may be a time gap between the administration of the two components.

In one embodiment, the invention relates to the use of a compound of Formula I or a pharmaceutically acceptable salt thereof and a compound useful in active or passive Tau immunotherapy in the manufacture of a medicament for the treatment of neurodegenerative or cognitive disorder, e.g. Alzheimer's disease.

In one embodiment, the invention provides a compound of Formula I or a pharmaceutically acceptable salt thereof and a compound useful in active or passive Tau immunotherapy for use in a method for the treatment of a neurodegenerative or cognitive disorder, e.g. Alzheimer's disease.

In one embodiment, the invention provides a pharmaceutical composition comprising a compound of Formula I or a pharmaceutically acceptable salt thereof and a compound useful in active or passive Tau immunotherapy and a pharmaceutically acceptable carrier.

Another paradigm to treat neurodegenerative and cognitive disorder, e.g. Alzheimer's disease is to target the Aβ peptides. It has been suggested that this can be achieved by either passive or active immunotherapy targeting Aβ peptides [J Neurosci, 34, 11621-11630, 2014; J Neurosci 33, 4923-4934, 2013]. In combination with compounds of the present invention this would attempt to target the same pathological mechanism via two different routes. Anti-Aβ antibodies (either injected directly into the patient or generated in the patient as a result of active immunotherapy) clear Aβ deposits in the brain, while further accumulation of Aβ peptide is blocked or reduced by the compounds of the present invention.

In one embodiment the invention provides a method for the treatment of a neurodegenerative or cognitive disorder, e.g. Alzheimer's disease, the method comprising the administration of a therapeutically effect amount of two components (1) a compound of Formula I or a pharmaceutically acceptable salt thereof and (2) a compound useful in active or passive Aβ peptide immunotherapy to a patient in need thereof. Said compound useful in passive Aβ peptide immunotherapy may be an anti-Aβ peptide antibody, such as gantenerumab, solanezumab, aducanumab or crenezumab. Said compound useful in active Aβ peptide immunotherapy may be a fragment of the Aβ peptide amino acid sequence which upon injection into a patient elicits anti-Aβ peptide antibodies in said patient. The administration according to this embodiment of the invention may be simultaneous, or there may be a time gap between the administration of the two components.

In one embodiment, the invention relates to the use of a compound of Formula I or a pharmaceutically acceptable salt thereof and a compound useful in active or passive Aβ peptide immunotherapy in the manufacture of a medicament for the treatment of neurodegenerative or cognitive disorder, e.g. Alzheimer's disease.

In one embodiment, the invention provides a compound of Formula I or a pharmaceutically acceptable salt thereof and a compound useful in active or passive Aβ peptide immunotherapy for use in a method for the treatment of a neurodegenerative or cognitive disorder, e.g. Alzheimer's disease.

In one embodiment, the invention provides a pharmaceutical composition comprising a compound of Formula I or a pharmaceutically acceptable salt thereof and a compound useful in active or passive Aβ peptide immunotherapy and a pharmaceutically acceptable carrier.

The NMDA (N-Methyl-D-Aspartate) receptor antagonist memantine and the acetylcholine esterase inhibitors donepezil, rivastigmine and galantamine are approved drugs for the treatment of Alzheimer's disease.

In one embodiment the invention provides a method for the treatment of a neurodegenerative or cognitive disorder, e.g. Alzheimer's disease, the method comprising the administration of a therapeutically effect amount of two components (1) a compound of Formula I or a pharmaceutically acceptable salt thereof and (2) an NMDA receptor antagonist or an acetylcholine esterase inhibitor to a patient in need thereof. The administration according to this embodiment of the invention may be simultaneous, or there may be a time gap between the administration of the two components.

In one embodiment, the invention relates to the use of a compound of Formula I or a pharmaceutically acceptable salt thereof and an NMDA receptor antagonist or an acetylcholine esterase inhibitor in the manufacture of a medicament for the treatment of neurodegenerative or cognitive disorder, e.g. Alzheimer's disease.

In one embodiment, the invention provides a compound of Formula I or a pharmaceutically acceptable salt thereof and an NMDA receptor antagonist or an acetylcholine esterase inhibitor for use in a method for the treatment of a neurodegenerative or cognitive disorder, e.g. Alzheimer's disease.

In one embodiment, the invention provides a pharmaceutical composition comprising a compound of Formula I or a pharmaceutically acceptable salt thereof and an NMDA receptor antagonist or an acetylcholine esterase inhibitor and a pharmaceutically acceptable carrier.

Seizures or epileptiform activity are also associated with Alzheimer's disease, including early stages of Alzheimer's disease, and treatment of said epileptic activity, which seeks to normalise hippocampal hyperactivity, may form part of an Alzheimer's disease treatment paradigm [JAMA Neurol, 70, 1158-1166, 2013; J Neurosci Res, 93, 454, 465, 2015; Neuron, 74, 647-474, 2012; Neurepsychpharm, 35, 1016-1025, 2010; CNS Neurosci Ther, 19, 871-881, 2013]. Useful antiepileptics include NMDA receptor antagonists and ion channel modulators, such as topiramate, levetiracetam and lamotrigine.

In one embodiment the invention provides a method for the treatment of a neurodegenerative or cognitive disorder, e.g. Alzheimer's disease, the method comprising the administration of a therapeutically effect amount of two components (1) a compound of Formula I or a pharmaceutically acceptable salt thereof and (2) an antiepileptic to a patient in need thereof. The administration according to this embodiment of the invention may be simultaneous, or there may be a time gap between the administration of the two components.

In one embodiment, the invention relates to the use of a compound of Formula I or a pharmaceutically acceptable salt thereof and an antiepileptic in the manufacture of a medicament for the treatment of neurodegenerative or cognitive disorder, e.g. Alzheimer's disease.

In one embodiment, the invention provides a compound of Formula I or a pharmaceutically acceptable salt thereof and an antiepileptic for use in a method for the treatment of a neurodegenerative or cognitive disorder, e.g. Alzheimer's disease.

In one embodiment, the invention provides a pharmaceutical composition comprising a compound of Formula I or a pharmaceutically acceptable salt thereof and an antiepileptic and a pharmaceutically acceptable carrier.

Emerging evidence suggests that inflammation has a causal role in Alzheimer's disease pathogenesis and that neuroinflammation is not a passive system activated by emerging β-amyloid deposits and neurofibrilary tangles, but also contributes to pathogenesis itself [Lancet Neurol, 14, 388-405, 2015; J Alz Dis, 44, 385-396, 2015; Neurol, 84, 2161-2168, 2015]. It follows from this that anti-inflammatory drugs, such as NSAID (non-steriod anti-inflammatory drugs), TNFα inhibitors, such as etanercept and p38 MAP kinase inhibitors, such as VX-745 (5-(2,6-Dichlorophenyl)-2-((2,4-difluorophenyl)thio)-6H-pyrimido[1,6-b]pyridazin-6-one) may be useful in the treatment of Alzheimer's disease.

In one embodiment the invention provides a method for the treatment of a neurodegenerative or cognitive disorder, e.g. Alzheimer's disease, the method comprising the administration of a therapeutically effect amount of two components (1) a compound of Formula I or a pharmaceutically acceptable salt thereof and (2) an anti-inflammatory drug to a patient in need thereof. The administration according to this embodiment of the invention may be simultaneous, or there may be a time gap between the administration of the two components.

In one embodiment, the invention relates to the use of a compound of Formula I or a pharmaceutically acceptable salt thereof and anti-inflammatory drug in the manufacture of a medicament for the treatment of neurodegenerative or cognitive disorder, e.g. Alzheimer's disease.

In one embodiment, the invention provides a compound of Formula I or a pharmaceutically acceptable salt thereof and an anti-inflammatory drug for use in a method for the treatment of a neurodegenerative or cognitive disorder, e.g. Alzheimer's disease.

In one embodiment, the invention provides a pharmaceutical composition comprising a compound of Formula I or a pharmaceutically acceptable salt thereof and an anti-inflammatory drug and a pharmaceutically acceptable carrier.

In addition, efficacy in the treatment of Alzheimer's disease has been demonstrated for Tau protein aggregation inhibitors, such as TRX-0237, also known as Methylene Blue, and SSRIs (Selective Serotonin Reuptake Inhibitor), such as citalopram [Behav Pharmacol, 26, 353-368, 2015; Sci Transl Med, 6(236re4), 2014].

In one embodiment the invention provides a method for the treatment of a neurodegenerative or cognitive disorder, e.g. Alzheimer's disease, the method comprising the administration of a therapeutically effect amount of two components (1) a compound of Formula I or a pharmaceutically acceptable salt thereof and (2) Tau protein aggregation inhibitor or an SSRI to a patient in need thereof. The administration according to this embodiment of the invention may be simultaneous, or there may be a time gap between the administration of the two components.

In one embodiment, the invention relates to the use of a compound of Formula I or a pharmaceutically acceptable salt thereof and a Tau protein aggregation inhibitor or an SSRI in the manufacture of a medicament for the treatment of neurodegenerative or cognitive disorder, e.g. Alzheimer's disease.

In one embodiment, the invention provides a compound of Formula I or a pharmaceutically acceptable salt thereof and a Tau protein aggregation inhibitor or an SSRI drug for use in a method for the treatment of a neurodegenerative or cognitive disorder, e.g. Alzheimer's disease.

In one embodiment, the invention provides a pharmaceutical composition comprising a compound of Formula I or a pharmaceutically acceptable salt thereof and a Tau protein aggregation inhibitor or an SSRI drug and a pharmaceutically acceptable carrier.

In one embodiment, a mammal is a human.

In one embodiment, the patient is a human patient.

### Pharmaceutically Acceptable Salts

The present invention also comprises salts of the present compounds, typically, pharmaceutically acceptable salts. Such salts include pharmaceutically acceptable acid addition salts. Acid addition salts include salts of inorganic acids as well as organic acids.

Pharmaceutically acceptable salts of a compound of Formula I are prepared e.g. in a conventional manner by treating a solution or suspension of a free base of Formula I with a molar equivalent of a pharmaceutically acceptable acid. Representative examples of suitable organic and inorganic acids are described below.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines (for example, 8-bromotheophylline and the like). Further examples of pharmaceutically acceptable inorganic or organic acid addition salts include the pharmaceutically acceptable salts listed in S. M. Berge, et al., J. Pharm. Sci., 1977, 66, 2.

Furthermore, the compounds of this invention may exist in unsolvated as well as in solvated forms with pharmaceutically acceptable solvents such as water, ethanol and the like.

The compounds of the present invention may have one or more asymmetric centres and it is intended that any optical isomers (i.e. enantiomers or diastereomers), as separated, pure or partially purified optical isomers and any mixtures thereof including racemic mixtures, i.e. a mixture of stereoisomers, are included within the scope of the invention.

The compounds of the present invention may exist exists in two stereo form, i.e. both of

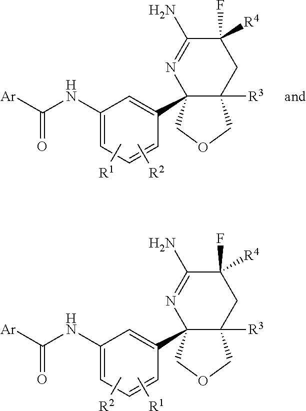

which are part of the invention.

In this context, it is understood that when specifying the enantiomeric form, then the compound is in enantiomeric excess, e.g. essentially in a pure form. Accordingly, one embodiment of the invention relates to a compound of the invention having an enantiomeric excess of at least 60%, at least 70%, at least 80%, at least 85%, at least 90%, at least 96%, preferably at least 98%.

Racemic forms may be resolved into the optical antipodes by known methods, for example, by separation of diastereomeric salts thereof with an optically active acid, and liberating the optically active amine compound by treatment with a base. Separation of such diastereomeric salts can be achieved, e.g. by fractional crystallization. The optically active acids suitable for this purpose may include, but are not limited to d- or l-tartaric, mandelic or camphorsulfonic acids. Another method for resolving racemates into the optical antipodes is based upon chromatography on an optically active matrix. The compounds of the present invention may also be resolved by the formation and chromatographic separation of diastereomeric derivatives from chiral derivatizing reagents, such as, chiral alkylating or acylating reagents, followed by cleavage of the chiral auxiliary. Any of the above methods may be applied either to resolve the optical antipodes of the compounds of the invention per se or to resolve the optical antipodes of synthetic intermediates, which can then be converted by methods described herein into the optically resolved final products which are the compounds of the invention.

In one aspect of the invention, the compound of the invention exists in racemic form

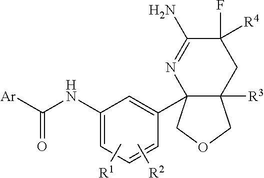

Additional methods for the resolution of optical isomers, known to those skilled in the art, may be used. Such methods include those discussed by J. Jaques, A. Collet and S. Wilen in Enantiomers, Racemates, and Resolutions, John Wiley and Sons, New York, 1981. Optically active compounds can also be prepared from optically active starting materials.

### Pharmaceutical Compositions

The present invention further provides a pharmaceutical composition comprising a compound of Formula I or a pharmaceutically acceptable salt thereof and a pharmaceutically acceptable carrier. The present invention also provides a pharmaceutical composition comprising a specific compound disclosed in the Experimental Section or a pharmaceutically acceptable salt thereof and a pharmaceutically acceptable carrier.

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 22^{th} Edition, Gennaro, Ed., Mack Publishing Co., Easton, Pa., 2013.

Pharmaceutical compositions for oral administration include solid dosage forms such as capsules, tablets, dragees, pills, lozenges, powders and granules. Where appropriate, the compositions may be prepared with coatings such as enteric coatings or they may be formulated so as to provide controlled release of the active ingredient such as sustained or prolonged release according to methods well known in the art. Liquid dosage forms for oral administration include solutions, emulsions, suspensions, syrups and elixirs. Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Other suitable administration forms include, but are not limited to, suppositories, sprays, ointments, creams, gels, inhalants, dermal patches and implants.

Typical oral dosages range from about 0.01 to about 100 mg/kg body weight per day.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of Formula I or a pharmaceutically acceptable salt thereof and a pharmaceutically acceptable carrier are readily administered in a variety of dosage forms suitable for the disclosed routes of administration. The formulations may conveniently be presented in unit dosage form by methods known in the art of pharmacy.

If a solid carrier is used for oral administration, the preparation may be tableted, placed in a hard gelatin capsule in powder or pellet form or it may be in the form of a troche or lozenge. The amount of solid carrier will vary widely but will range from about 25 mg to about 1 g per dosage unit. If a liquid carrier is used, the preparation may be in the form of a syrup, emulsion, soft gelatin capsule or sterile injectable liquid such as an aqueous or non-aqueous liquid suspension or solution.

### EXAMPLES

The compounds of the present invention of the general Formula I, wherein R¹, R², R³, R⁴ and Ar are as defined above can be prepared by the methods outlined in the following reaction schemes 1-6 and in the Examples. In the described methods, it is possible to make use of variants or modifications, which are themselves known to chemists skilled in the art or could be apparent to the person of ordinary skill in this art. Furthermore, other methods for preparing compounds of the invention will be readily apparent to the person skilled in the art in light of the following reaction schemes and Examples.

For example, Scheme 2 describes the use of selective protecting groups during the synthesis of the compounds of the invention. One skilled in the art would be able to select the appropriate protecting group for a particular reaction. Moreover, it may be necessary to incorporate protection and deprotection strategies for substituents such as amino, amido, keto and hydroxyl groups in the synthetic methods described below to synthesize the compounds of Formula I. Methods for protection and deprotection of such groups are well known in the art, and may be found in T. Green, et al., Protective Groups in Organic Synthesis, 1991, 2^{nd} Edition, John Wiley & Sons, New York.

For compounds, which can exist as a mixture or equilibrium between two or more tautomers, only one tautomer is represented in the schemes, although it may not be the most stable tautomer. For compounds, which can exist in enantiomeric, stereoisomeric or geometric isomeric forms their geometric configuration is specified; otherwise the structure represents a mixture of stereoisomers.

Analytical LC-MS data was obtained using the following methods.

Method A:

LC-MS was run on Waters Aquity UPLC-MS consisting of Waters Aquity including column manager, binary solvent manager, sample organizer, PDA detector (operating at 254 nM), ELS detector, and SQ-MS equipped with APPI-source operating in positive ion mode.

LC-conditions: The column was Acquity UPLC BEH C18 1.7 μm; 2.1×150 mm operating at 60° C. with 0.6 mL/min of a binary gradient consisting of water+0.05% trifluoroacetic acid (A) and acetonitrile+5% water+0.03% trifluoroacetic acid (B). Gradient: 0.00 min: 10% B; 3.00 min: 99.9% B; 3.01 min: 10% B; 3.60 min: 10% B. Total run time: 3.60 min.

Method B:

LC-MS was run on Waters Acquity UPLC-MS consisting of Waters Acquity including column manager, binary solvent manager, sample organizer, PDA detector (operating at 254 nm), ELS detector, and TQ-MS equipped with APPI-source operating in positive ion mode.

LC-conditions: The column was Acquity UPLC BEH C18 1.7 μm; 2.1×50 mm operating at 60° C. with 1.2 mL/min of a binary gradient consisting of water+0.05% trifluoroacetic acid (A) and acetonitrile+5% water+0.05% trifluoroacetic acid (B). Gradient: 0.00 min: 10% B; 1.00 min: 100% B; 1.01 min: 10% B; 1.15 min: 10% B. Total run time: 1.15 min.

Method C:

An Agilent 1200 LCMS system with ELS detector was used. Column: Agilent TC-C18 5 μm;

2.1×50 mm; Column temperature: 50° C.; Solvent system: A=water/trifluoroacetic acid (99.9:0.1) and B=acetonitrile/trifluoroacetic acid (99.95:0.05); Method: Linear gradient elution with A:B=99:1 to 0:100 in 4.0 minutes and with a flow rate of 0.8 mL/min.

Method D: An Agilent 1200 LCMS system with ELS detector was used. Column: XBridge ShieldRP18, 5 μm, 50×2.1 mm; Column temperature: 40° C.; Solvent system: A=water/conc. NH₃ (aq) (99.95:0.05) and B=acetonitrile; Method: Linear gradient elution with A:B=95:5 to 0:100 in 3.4 minutes and with a flow rate of 0.8 mL/min.

Method E:

An Agilent 6100 LCMS system was used. Column: Xbrige Shield RP-18.5 um, 2.1*50 mm;

Column temperature: 30° C.; Solvent system: A:=water (1 L)/conc. NH₃ (aq) (0.5 mL) and B=acetonitrile; Method: Linear gradient elution with B %=10% at 0 min, 80% at 2 min, 80% at 2.48 min, 10% at 2.49 min, 10% at 3 min and with a flow rate of 1 mL/min.

Method F:

An Agilent 6100 LCMS system was used. Column: MERCK,RP-18e 25-2 mm; Column temperature: 50° C.; Solvent system: A:water (4 L)+TFA (1.5 mL) and B:acetonitrile (4 L)+TFA (0.75 mL); Method: Linear gradient elution with B %=5% at 0 min, 95% at 0.7 min, 95% at 1.1 min, 5% at 1.11 min, 5% at 1.5 min and with a flow rate of 1.5 mL/min.

¹H NMR spectra were recorded at 600 MHz on a Bruker Avance AV-III-600 instrument or at 400 MHz on a Bruker Avance AV-III-400 instrument or a Varian 400 instrument. Chemical shift values are expressed in ppm-values relative. The following abbreviations are used for multiplicity of NMR signals: s=singlet, d=doublet, t=triplet, q=quartet, dd=double doublet, ddd=double double doublet, dt=double triplet, br=broad, and m=multiplet.

Compounds of the general formulae VIa and VIb may be prepared as shown in Scheme 1.

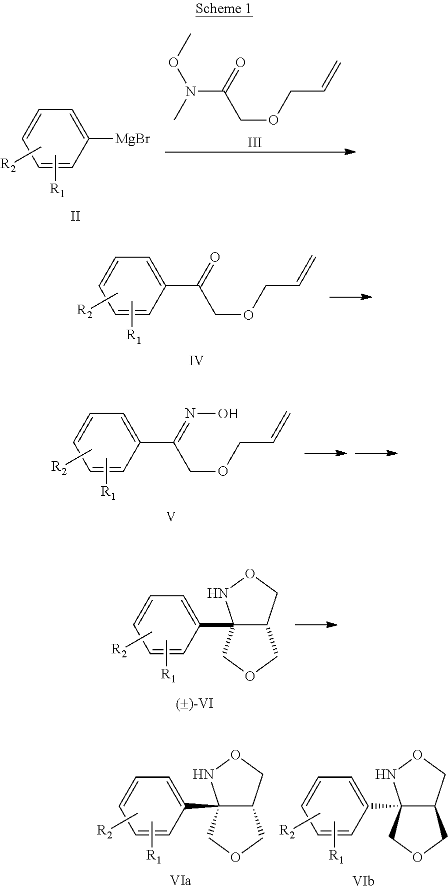

where R¹ and R² as defined under Formula I

Compounds of the general formula IV (Scheme 1) may be prepared by reacting compounds of the general formula II with Weinreb amide III. A condensation reaction between compounds of the general formula IV and hydroxyl amine gives compounds of the general formula V. An intramolecular cycloaddition reaction of compounds of the general formula V gives a racemic mixture of compounds of the general formula (±)-VI, which can be separated into the two enantiomers VIa and VIb by chromatographic methods such as SFC (supercritical fluid chromatography) with a optically pure chiral stationary phase.

Compounds of the general formula VIII may be prepared as shown in Scheme 2.

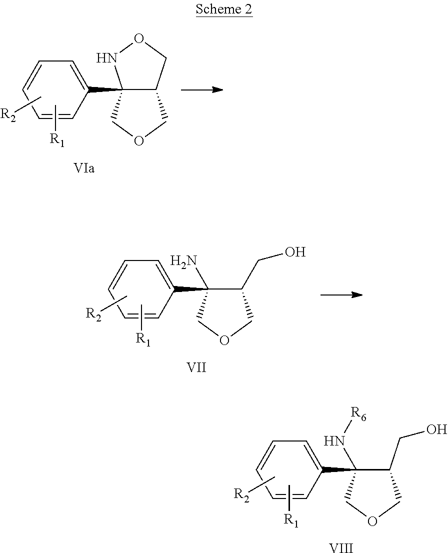

where R¹ and R² are as defined under formula I and R⁶ is an amine protection groups such as a tert-butoxy carbonyl group or a benzyloxy carbonyl group.

Compounds of the general formula VII (Scheme 2) may be prepared by reduction of the N—O bond of compounds of the general formula VIa with a reducing agent such as lithium aluminum hydride. The amine moiety can then be protected with an amine protection group such as a tert-butoxy carbonyl group or a benzyloxy carbonyl group to give compounds of the general formula VIII.

Compounds of the general formulae XXa and XXb may be prepared as shown in Scheme 3.

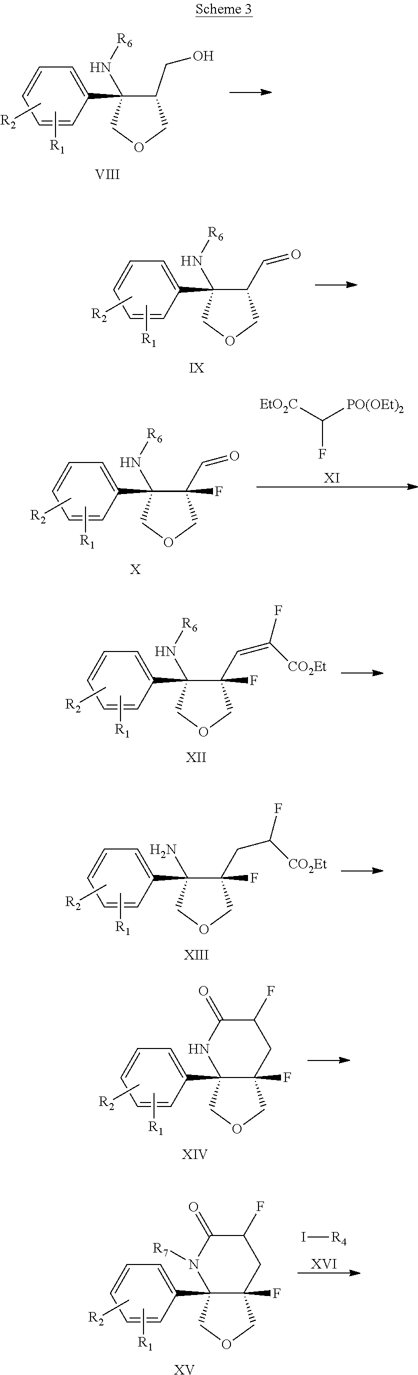

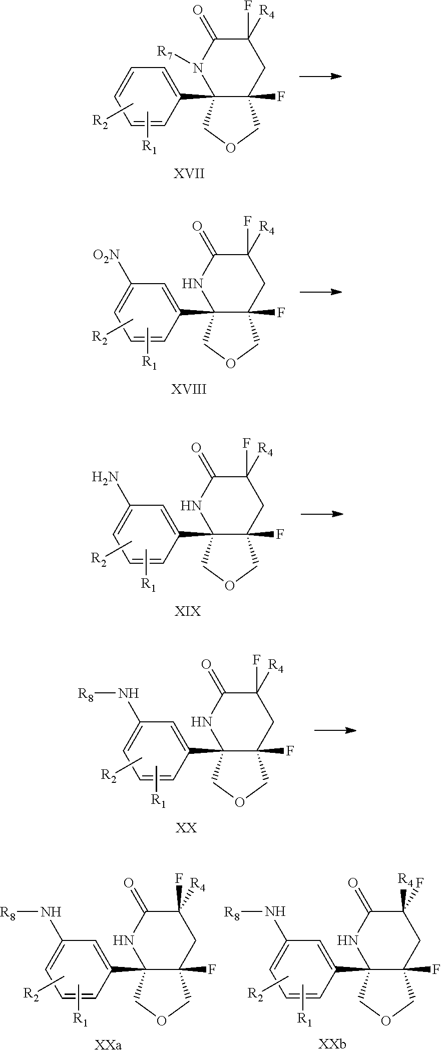

where R¹, R² and R⁴ are as defined under Formula I and R⁶ is an amine protection groups such as a benzyloxy carbonyl group, R⁷ is a protection groups such as a tert-butoxy carbonyl group and R⁸ is a protection groups such as a tert-butoxy carbonyl group.

Compounds of the general formula IX (Scheme 3) may be prepared oxidation of compounds of the general formula VIII with an oxidant such as DMP (Dess Martin periodinane). Compounds of the general formula X can be prepared by an organocatalytic fluorination reaction using a catalyst such as (S)-2-(bis(3,5-bis(trifluoromethyl)phenyl)((trimethylsilyl)oxy)methyl)pyrrolidine and a fluorinating reagent such as NFSi (N-fluoro-N-(phenylsulfonyl)benzenesulfonamide). Compounds of the general formula XII are then prepared by a Horner Wadsworth Emmons reaction between compounds of the general formula X and ethyl 2-(diethoxyphosphoryl)-2-fluoroacetate XI in the presence of a base such as triethylamine and lithium chloride. Reduction of the double bond of compounds of the general formula XII in the presence of a catalysts such as palladium on carbon under an atmosphere of hydrogen also removes the nitrogen protection group when the latter is a benzyloxy carbonyl group to give compounds of the general formula XIII. Compounds of the general formula XIII can be ring-closed to give compounds of the general formula XIV by treatment with methanol and a base such as potassium carbonate. After protection of the amide group with a protection group such as a tert-butoxy carbonyl group, compounds of the general formula XV can be alkylated by treatment with a strong base such as LiHMDS (lithium bis(trimethylsilyl)amide) followed by treatment with compounds of the general formula XVI to form compounds of the general formula XVII as a mixture of diastereomers. Treatment of compounds of the general formula XVII with sulfuric acid and nitric acid leads to removal of the amide protection group and nitration to give compounds of the general formula XVIII. Reduction of the nitro moiety of compounds of the general formula XVIII gives compounds of the general formula XIX, which after protection of the amino moiety gives compounds of the general formula XX as a mixture of diastereomers. The mixture of diastereomers can at this stage be separated to give compounds of the general formula XXa and compounds of the general formula XXb.

Compounds of the general formulae XXIIa and XXIIb may be prepared as shown in Scheme 4.

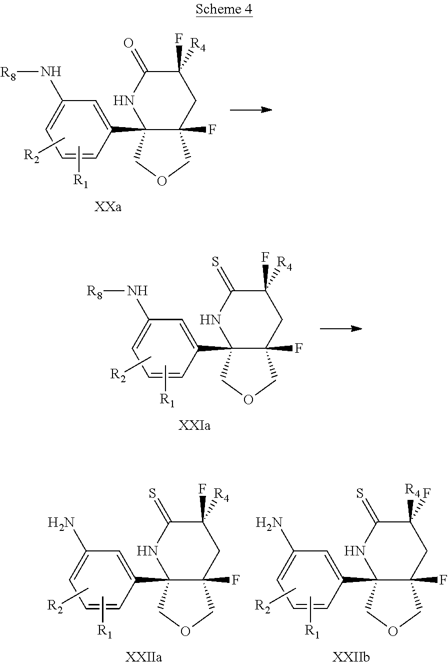

where R¹, R² and R⁴ are as defined under Formula I and R⁸ is an amine protection groups such as a tert-butoxy carbonyl group.

Compounds of the general formula XXIa (Scheme 4) can be obtained by treating compounds of the general formula XXa with a reagent such as Lawesson's reagent (2,4-bis(4-methoxyphenyl)-1,3,2,4-dithiadiphosphetane-2,4-disulfide). Deprotection of the amine moiety of compounds of the general formula XIXa gives compounds of the general formula XXIIa. Compounds of the general formula XXIIb can be obtained in a similar way from compounds of the general formula XXb.

Compounds of the general formulae XXIIc and XXIId may be prepared as shown in Scheme 5.

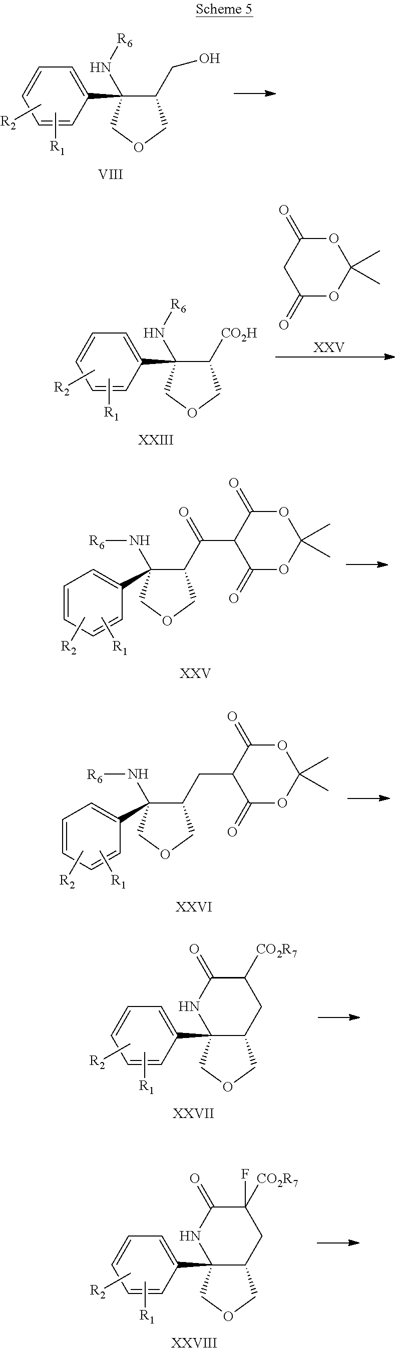

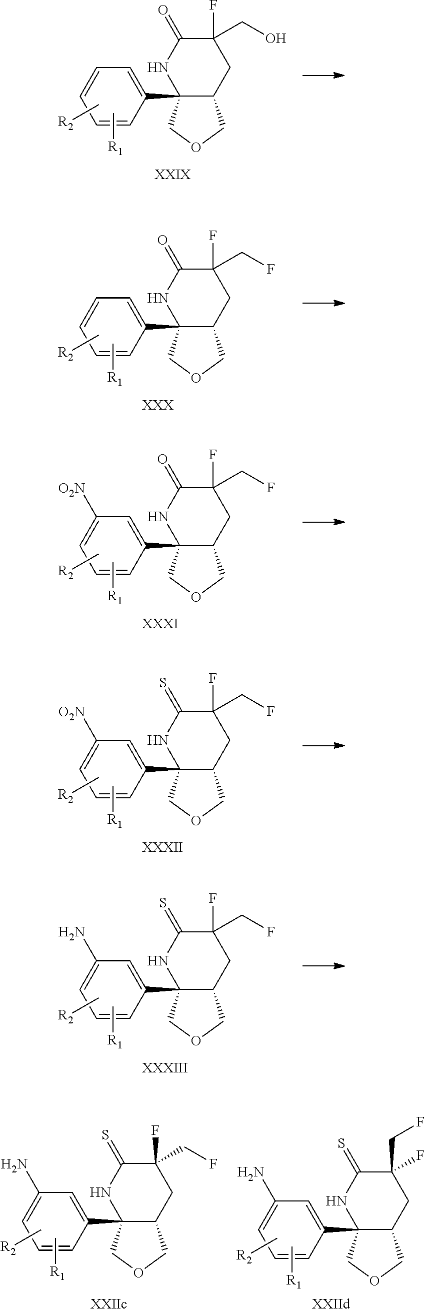

where R¹ and R² are as defined under Formula I, R⁶ is an amine protection groups such as a tert-butoxy carbonyl group and R⁷ is an alkyl group such as methyl or ethyl.

Compounds of the general formula XXIII (Scheme 5) may be prepared by oxidation of compounds of the general formula VIII. Compounds of the general formula XXV may be prepared by reaction of compounds of the general formula XXIII with Meldrum's acid (XXIV) in the presence of a coupling reagent such as EDC (1-ethyl-3-(3-dimethylaminopropyl)carbodiimide). Reduction of compounds of the general formula XXV with a reductant such as sodium borohydride in acetic acid gives compounds of the general formula XXVI. Treatment of compounds of the general formula XXVI with an acid such as hydrochloric acid in an alcohol solvent such as methanol or ethanol followed by treatment with a base such as triethylamine gives compounds of the general formula XXVII. Compounds of the general formula XXVII can be fluorinated with a reagent such as NFSi (N-fluorobenzenesulfonimide) to give compounds of the general formula XXVIII, which after reduction with a regent such as sodium borohydride gives compounds of the general formula XXIX. Activation of the alcohol moiety of compounds of the general formula XXIX with a regent such as NfF (1,1,2,2,3,3,4,4,4-nonafluorobutane-1-sulfonyl fluoride) in the presence of a base such as triethylamine followed by a substitution reaction with a regent such as TBAF (tetrabutylammonium fluoride) gives compounds of the general formula XXX. Treatment of compounds of the general formula XXX with sulfuric acid and nitric acid leads to nitration to give compounds of the general formula XXXI. Compounds of the general formula XXXII can be obtained by treating compounds of the general formula XXXI with a reagent such as Lawesson's reagent (2,4-bis(4-methoxyphenyl)-1,3,2,4-dithiadiphosphetane-2,4-disulfide). Reduction of the nitro moiety of compounds of the general formula XXXII followed by separation of the two diastereomers of compounds of the general formula XXXIII gives the compounds of the general formulae XXIIc and XXIId. Compounds of the general formula I may be prepared as shown in Scheme 6.

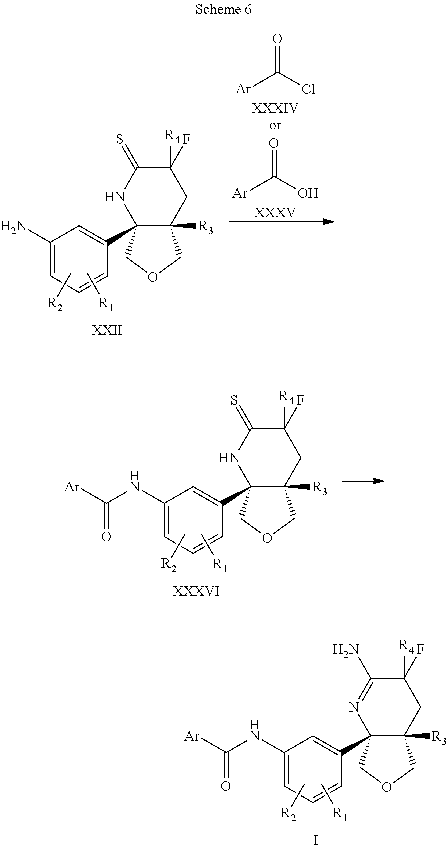

where R¹, R², R³, R⁴ and Ar are as defined under Formula I.

Compounds of the general formula XXXVI (Scheme 6) may be prepared by reacting compounds of the general formula XXIII with a carboxylic acid chloride of general formula XXXIV or by reaction with a carboxylic acid of general formula XXXV using procedures known to chemists skilled in the art. Carboxylic acid chlorides of general formula XXXIV and carboxylic acids of general formula XXXV are either commercially available or can be synthesized by methods described in the literature. Treatment of compounds of the general formula XXXVI with ammonia gives compounds of the general formula I. In some cases, the addition of an oxidizing reagent such as tert-butyl hydroperoxide might be necessary to facilitate the reaction.

### PREPARATION OF INTERMEDIATES

### Intermediate I-1: 2-(allyloxy)-N-methoxy-N-methylacetamide

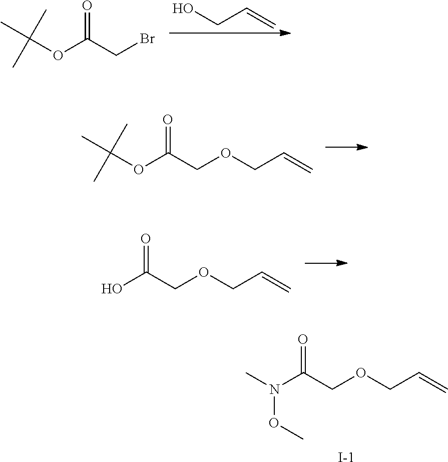

Tetrabutylammonium hydrogen sulfate (9.41 g, 27.7 mmol) was added to a solution of sodium hydroxide (305 mL, 3296 mmol, 10.8 molar, water) in water (100 mL) and toluene (280 mL). Prop-2-en-1-ol (16.09 g, 18.84 mL, 277 mmol) was added. The reaction mixture was cooled to 0° C. Tert-butyl 2-bromoacetate (80 g, 60.6 mL, 410 mmol) was added slowly. The reaction mixture was stirred at room temperature overnight. 250 mL water was added. The mixture was extracted with heptane. The organic phase was washed with brine, dried over magnesium sulfate and concentrated in vacuo to give tert-butyl 2-(allyloxy)acetate (47.4 g, 99% yield). Used in the next step without further purification.

tert-Butyl 2-(allyloxy)acetate (47.4 g, 275 mmol) in toluene (87 g, 100 mL, 939 mmol) was cooled to 0° C. Formic acid (200 mL) was added at a rate to keep internal temperature <10° C. The reaction mixture was stirred at 55° C. for overnight. The reaction mixture was concentrated in vacuo followed by azeotropic removal of residual formic acid with three portions of toluene (3×100 mL) to give 2-(allyloxy)acetic acid (31.2 g 97% yield). Used in the next step without further purification.

N,N-carbonyldiimidazole (47.8 g, 295 mmol) was added to 2-(allyloxy)acetic acid (31.15 g, 268 mmol) in dichloromethane (170 mL) in portions at 0° C. The reaction mixture was stirred at 0° C. for 1 hour. N,O-dimethylhydroxylamine hydrochloride (30.1 g, 309 mmol) was added in 5 portions. The reaction mixture was allowed to warm to room temperature and was stirred for 3 hours. Imidazole (4.57 g, 67.1 mmol) was added. The reaction mixture was stirred at room temperature for 1 hour. Water (250 mL) was added. The organic phase was washed with 1 N aq. HCl (2×250 mL), brine and dried over magnesium sulfate. The solution was filtered through silica gel (eluted with ethyl acetate/heptane 1:1) and concentrated in vacuo to give 2-(allyloxy)-N-methoxy-N-methylacetamide (30.7 g, 72% yield). Used in the next step without further purification.

### Intermediate I-2: 2-(allyloxy)-1-(2,3-difluorophenyl)ethan-1-one

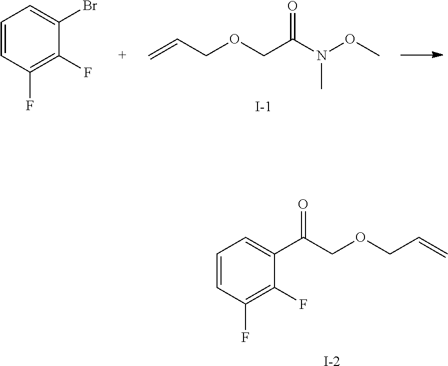

To a solution of 1-bromo-2,3-difluorobenzene (48.0 g, 249 mmol) in THF (1.00 L) was added dropwise ethylmagnesium bromide (3 M, 83 mL) at −78° C. under N₂ with stirring. After addition, the mixture was stirred at 0° C. for 1 h, and then 2-allyloxy-N-methoxy-N-methyl-acetamide (51.5 g, 323 mmol) in THF (50 mL) was added dropwise at 0° C. The yellow solution was stirred at 0° C. for 1 h. The reaction mixture was quenched by addition of saturated aqueous NH₄Cl (200 mL) at 0° C., and then filtered. The filtrate was concentrated under reduced pressure. The residue was diluted with saturated aqueous NH₄Cl (200 mL) and extracted with EtOAc (200 mL×2). The combined organic layers were washed with brine, dried over Na₂SO₄, filtered, concentrated under reduced pressure and purified by column chromatography (silica gel, Gradient elution: ethyl acetate in petroleum ether=0%˜5%) to afford 2-(allyloxy)-1-(2,3-difluorophenyl)ethan-1-one (33.0 g, 62.5% yield).

### Intermediate I-3: 2-(allyloxy)-1-(2,3-difluorophenyl)ethan-1-one oxime

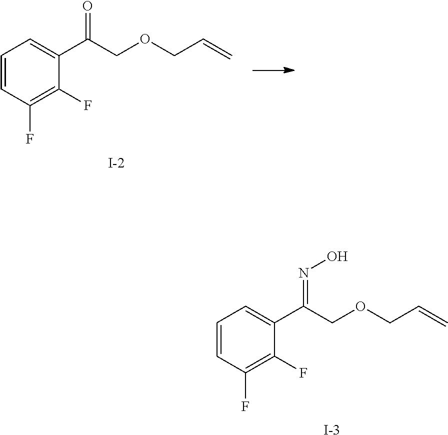

To a solution of I-2 (33.0 g, 156 mmol, 1.00 eq) in ethanol (500 mL) was added NH₂OH.HCl (13.0 g, 187 mmol, 1.20 eq) and sodium acetate (19.1 g, 233 mmol, 1.50 eq). The yellow solution was stirred at 60° C. for 2 h. The reaction mixture was cooled down to 20° C. and filtered; the filtrate was concentrated under reduced pressure to remove ethanol. The residue was diluted with water (100 mL) and extracted with EtOAc (100 mL×2). The combined organic layers were washed with brine, dried over Na₂SO₄, filtered and concentrated under reduced pressure to give 2-(allyloxy)-1-(2,3-difluorophenyl)ethan-1-one oxime (36.09 g, crude), which was used for next step directly without further purification.

### Intermediate I-4: 6a-(2,3-difluorophenyl)tetrahydro-1H,3H-furo[3,4-c]isoxazole

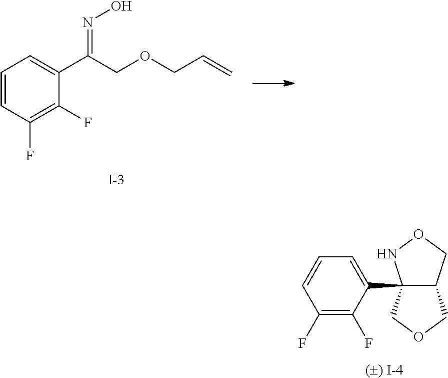

To a solution of 2-(allyloxy)-1-(2,3-difluorophenyl)ethan-1-one oxime (36.1 g, 159 mmol, 1.00 eq) in xylene (500 mL) was added hydroquinone (3.60 g, 32.7 mmol, 0.21 eq). The yellow solution was stirred at 120° C. for 24 hours. The mixture was cooled down to 20° C. and filtered. The filtrate was concentrated under vacuo to remove xylene. The residue was washed with MTBE (20 mL), filtered, dried under vacuo to afford I-4 (11.69 g, 32% yield) as a white solid. ¹H NMR (CDCl₃ 400 MHz TMS): δ 7.67 (bs, 1H), 7.15-7.06 (m, 2H), 5.11 (s, 1H), 4.54 (bs, 1H), 4.20-4.14 (m, 1H), 4.00 (d, J=9.6 Hz, 1H), 3.93-3.85 (m, 2H), 3.58 (t, J=8.0 Hz, 1H), 3.43 (q, J=7.2 Hz, 1H).

### Intermediate I-5: 6a-(2-fluorophenyl)tetrahydro-1H,3H-furo[3,4-c]isoxazole

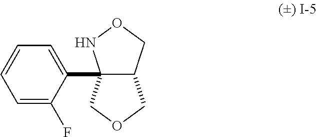

6a-(2-fluorophenyl)tetrahydro-1H,3H-furo[3,4-c]isoxazole was prepared using a similar procedure as was used for the synthesis of I-4 with intermediates corresponding to I-2 and I-3. The intermediate corresponding to I-2 was synthesized from 1-bromo-2-fluorobenzene and I-1.

### Intermediate I-6 (+)-(3aS,6aS)-6a-(2,3-difluorophenyl)tetrahydro-1H,3H-furo[3,4-c]isoxazole

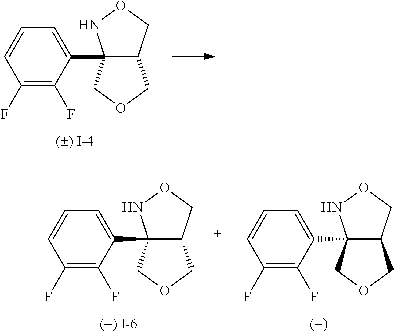

The two enantiomers of I-4 (±) 6a-(2,3-difluorophenyl)tetrahydro-1H,3H-furo[3,4-c]isoxazole (23.38 g) were separated by SFC (supercritical fluid chromatography). The SFC separation condition: Instrument: SFC-6. Column: AD (250 mm×50 mm, 10 um). Mobile phase: A: Supercritical CO₂, B: Base-IPA, A:B=75:25 at 200 mL/min. Column Temp: 40° C. Nozzle Pressure: 100 Bar. Nozzle Temp: 60° C. Evaporator Temp: 20° C. Trimmer Temp: 25° C. Wavelength: 220 nm.

(+)-I-6: (3aS,6aS)-6a-(2,3-difluorophenyl)tetrahydro-1H,3H-furo[3,4-c]isoxazole 10.41 g was obtained. ¹H NMR (DMSO-d₆ 400 MHz TMS): δ 7.62-7.52 (m, 1H), 7.41-7.30 (m, 1H), 7.24-7.12 (m, 1H), 6.23 (s, 1H), 4.31 (bs, 1H), 4.01-3.84 (m, 4H), 3.67 (dd, J=10.0, 2.4 Hz, 1H), 3.45 (bs, 1H). SFC: t_{R}=1.40. [α]²⁰,_{D}+19.87 (c=0.20, ethanol).

### Intermediate I-7: (+)-(3aS,6aS)-6a-(2-fluorophenyl)tetrahydro-1H,3H-furo[3,4-c]isoxazole

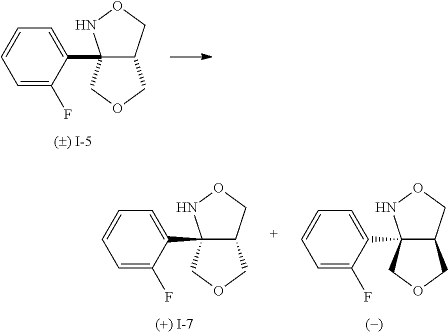

The two enantiomers of I-5 (±) 6a-(2-fluorophenyl)tetrahydro-1H,3H-furo[3,4-c]isoxazole (21.6 g) were separated by SFC (supercritical fluid chromatography). The SFC separation condition: Column: AD (250 mm×50 mm, 10 um). Mobile phase: A: Supercritical CO₂, B: Neu-EtOH, A:B=55:45 at 200 mL/min. Column Temp: 38° C. Nozzle Pressure: 100 Bar. Nozzle Temp: 60° C. Evaporator Temp: 20° C. Trimmer Temp: 25° C. Wavelength: 220 nm. Two peaks were collected and concentrated to give I-7 (+)-(3aS,6aS)-6a-(2-fluorophenyl)tetrahydro-1H,3H-furo[3,4-c]isoxazole (9.53 g, 85% yield) and (−)-(3aR,6aR)-6a-(2-fluorophenyl)tetrahydro-1H,3H-furo[3,4-c]isoxazole (9.53 g, 84% yield).

I-7 (3aS,6aS)-6a-(2-fluorophenyl)tetrahydro-1H,3H-furo[3,4-c]isoxazole: ¹H NMR (DMSO-d6, 400 MHz, TMS): δ 7.78 (t, J=7.7 Hz, 1H), 7.38-7.28 (m, 1H), 7.25-7.11 (m, 2H), 6.13 (s, 1H), 4.40-4.22 (m, 1H), 4.01-3.89 (m, 2H), 3.86 (dd, J=2.8, 9.6 Hz, 1H), 3.66 (dd, J=2.9, 9.9 Hz, 1H), 3.52-3.39 (m, 1H), 3.32-3.24 (m, 1H). SFC: t_{R}=3.02 min. [α]²⁰,_{D}+29.14 (589 nm, c=0.103, ethanol).

### Intermediate I-8a: ((3R,4S)-4-amino-4-(2-fluorophenyl)tetrahydrofuran-3-yl)methanol

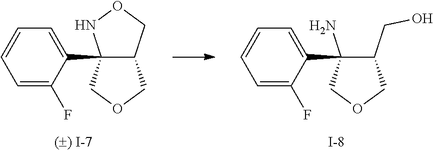

85 mL Lithium aluminum hydride in THF (85 mmol) was added to 100 mL THF and the mixture was cooled to −5° C. I-7 (+)-(3aS,6aS)-6a-(2-fluorophenyl)tetrahydro-1H,3H-furo[3,4-c]isoxazole in 100 mL THF was added dropwise. The reaction mixture was stirred at 0° C. for 1 hour then at room temperature for 30 minutes. The reaction mixture was cooled to 0° C. and was quenched by adding: 6.5 mL water. The reaction mixture was stirred at room temperature for 15 minutes. 3.24 mL 15% NaOH was added. The reaction mixture was stirred at room temperature for 15 minutes. 16.2 mL Water was added. The reaction mixture was stirred at room temperature for 15 minutes. 25 g Sodium sulfate was added. The reaction mixture was stirred at room temperature for 90 minutes, filtered through celite and concentrated in vacuo. Used in the next step without further purification.

¹H NMR (600 MHz, CDCl₃) δ 7.53 (td, J=8.1, 1.7 Hz, 1H), 7.33-7.27 (m, 1H), 7.19-7.14 (m, 1H), 7.09 (ddd, J=12.4, 8.2, 1.3 Hz, 1H), 4.14 (dd, J=9.1, 0.9 Hz, 1H), 4.07 (t, J=8.7 Hz, 1H), 4.00-3.90 (m, 3H), 3.83 (dd, J=11.8, 6.5 Hz, 1H), 2.89-2.81 (m, 1H).

I-8b ((3R,4S)-4-amino-4-(2,3-difluorophenyl)tetrahydrofuran-3-yl)methanol was prepared in a similar way from (+)-I-6 (+)-(3aS,6aS)-6a-(2,3-difluorophenyl)tetrahydro-1H,3H-furo[3,4-c]isoxazole

### Intermediate I-9: benzyl ((3S,4R)-3-(2-fluorophenyl)-4-(hydroxymethyl)tetrahydrofuran-3-yl)carbamate

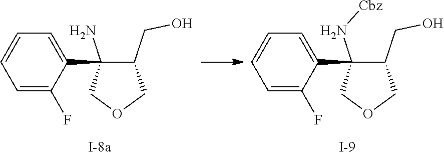

Benzyl chloroformate (7.69 g, 42.8 mmol) was added to a mixture of sodium carbonate (4.54 g, 42.8 mmol) and I-8a ((3R,4S)-4-amino-4-(2-fluorophenyl)tetrahydrofuran-3-yl)methanol (9.05 g, 42.8 mmol) in THF (160 mL) and water (16 mL) at 0° C. The reaction mixture was stirred at room temperature overnight. A 1:3 mixture of saturated sodium bicarbonate (aq):water was added.

The mixture was extracted with ethyl acetate and THF. The combined organic phases were washed with brine, dried over magnesium sulfate and concentrated in vacuo. The crude material was purified via flash chromatography on silica gel to give I-9 benzyl ((3S,4R)-3-(2-fluorophenyl)-4-(hydroxymethyl)tetrahydrofuran-3-yl)carbamate (14.0 g 94% yield) ¹H NMR (600 MHz, CDCl₃) δ 7.76 (t, J=7.9 Hz, 1H), 7.39-7.27 (m, 6H), 7.15 (t, J=7.5 Hz, 1H), 7.04 (ddd, J=12.3, 8.1, 1.2 Hz, 1H), 6.37 (bs, 1H), 5.02 (dd, J=33.8, 12.3 Hz, 2H), 4.25 (q, J=9.7 Hz, 2H), 4.05 (t, J=8.6 Hz, 1H), 3.83-3.71 (m, 2H), 3.59 (t, J=8.2 Hz, 1H), 3.24-3.12 (m, 2H).

### Intermediate I-10: benzyl ((3S,4S)-3-(2-fluorophenyl)-4-formyltetrahydrofuran-3-yl)carbamate

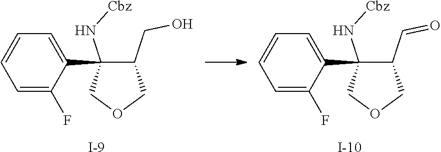

DMP (Dess-Martin Periodinane) (27.5 g, 64.7 mmol) was added to I-9: benzyl ((3S,4R)-3-(2-fluorophenyl)-4-(hydroxymethyl)tetrahydrofuran-3-yl)carbamate in dichloromethane (67 mL). The reaction mixture was stirred at room temperature for 2½ hours. Sodium thiosulfate (38 g) in water and saturated sodium bicarbonate (aq) were added. The reaction mixture was stirred at room temperature for 1½ hours. The mixture was extracted with ethyl acetate. The organic phase was washed with brine, dried over magnesium sulfate and concentrated in vacuo to give I-10 benzyl ((3S,4S)-3-(2-fluorophenyl)-4-formyltetrahydrofuran-3-yl)carbamate (15.4 g). Used in next step without any further purification.

¹H NMR (600 MHz, CDCl₃) δ 9.88 (s, 1H), 7.55-7.48 (m, 1H), 7.39-7.23 (m, 6H), 7.21-7.15 (m, 1H), 7.09 (dd, J=11.9, 8.4 Hz, 1H), 5.89 (s, 1H), 5.04-4.89 (m, 2H), 4.41-4.33 (m, 2H), 4.24 (d, J=7.9 Hz, 1H), 4.16-4.07 (m, 1H), 3.82-3.76 (m, 1H).

### Intermediate I-11: (4aR,7aS)-3,4a-difluoro-7a-(2-fluorophenyl)hexahydrofuro[3,4-b]pyridin-2(1H)-one

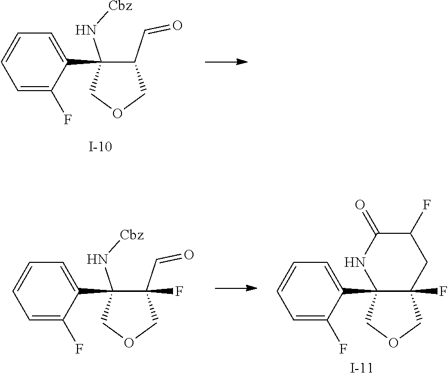

(S)-2-(bis(3,5-bis(trifluoromethyl)phenyl)((trimethylsilyl)oxy)methyl)pyrrolidine (2.26 g, 3.79 mmol) was added to benzyl ((3S,4S)-3-(2-fluorophenyl)-4-formyltetrahydrofuran-3-yl)carbamate (5.2 g, 15.14 mmol) in tert-butyl methyl ether (125 mL). The reaction mixture was stirred at room temperature for 15 minutes. NFSi (N-fluoro-N-(phenylsulfonyl)benzenesulfonamide) (6.69 g, 21.2 mmol) was added and the reaction mixture was stirred at 40° C. for 6 hours. Then (S)-2-(bis(3,5-bis(trifluoromethyl)phenyl)((trimethylsilyl)oxy)methyl)pyrrolidine (1 g, 1.67 mmol) was added. The reaction mixture was stirred at room temperature overnight. Triethylamine (11.60 mL, 83 mmol), ethyl 2-(diethoxyphosphoryl)-2-fluoroacetate (8.19 mL, 36.3 mmol, 90%), lithium chloride (1.052 g, 24.82 mmol) and acetonitrile (125 mL) were added. The reaction mixture was stirred at room temperature overnight. The reaction mixture was filtered and concentrated in vacuo. The crude product was purified by flash chromatography on silica gel (eluent: heptane/ethyl acetate). The intermediate was dissolved in methanol (70 mL). Palladium on carbon (10%, 0.6 g) was added and the reaction mixture was stirred under 2.8 bar hydrogen pressure at room temperature overnight. The reaction mixture was filtered and potassium carbonate (1.44 g, 10.4 mmol) was added. The reaction mixture was stirred at room temperature overnight and concentrated in vacuo. Water was added and the mixture was extracted with ethyl acetate. The organic phase was washed with brine, dried over magnesium sulfate and concentrated in vacuo. The crude product was purified by flash chromatography on silica gel (eluent: heptane/ethyl acetate) to give (4aR,7aS)-3,4a-difluoro-7a-(2-fluorophenyl)hexahydrofuro[3,4-b]pyridin-2(1H)-one as a mixture of diastereomers (1.72 g 41.9% yield). LC-MS (m/z) 272.1 (MH⁺); t_{R}=0.75 (Method B).

### Intermediate I-12: tert-butyl (4aR,7aS)-3,4a-difluoro-7a-(2-fluorophenyl)-2-oxohexahydrofuro[3,4-b]pyridine-1(2H)-carboxylate

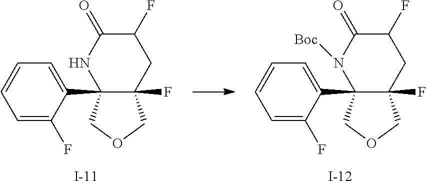

Di-tert-butyl dicarbonate (1.64 g, 7.51 mmol) and DMAP (N,N-dimethylpyridin-4-amine) (0.039 g, 0.32 mmol) were added to (4aR,7aS)-3,4a-difluoro-7a-(2-fluorophenyl)hexahydrofuro[3,4-b]pyridin-2(1H)-one (1.72 g, 6.34 mmol) in acetonitrile (100 mL). The reaction mixture was stirred at room temperature overnight and concentrated in vacuo. The crude product was purified by flash chromatography on silica gel (eluent: heptane/ethyl acetate) to give tert-butyl (4aR,7aS)-3,4a-difluoro-7a-(2-fluorophenyl)-2-oxohexahydrofuro[3,4-b]pyridine-1(2H)-carboxylate as a mixture of diastereomers (1.93 g, 82% yield). LC-MS (m/z) 272.1 (MH⁺-Boc); t_{R}=0.46 (Method B)

### Intermediates I-13: tert-butyl (3R,4aR,7aS)-3,4a-difluoro-7a-(2-fluorophenyl)-3-methyl-2-oxohexahydrofuro[3,4-b]pyridine-1(2H)-carboxylate and I-14: tert-butyl (3S,4aR,7aS)-3,4a-difluoro-7a-(2-fluorophenyl)-3-methyl-2-oxohexahydrofuro[3,4-b]pyridine-1(2H)-carboxylate

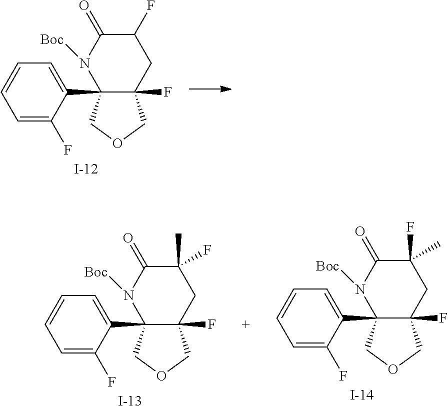

LiHMDS (lithium bis(trimethylsilyl)amide) (1M in THF, 28.5 mL, 28.5 mmol) was added dropwise to I-12 tert-butyl (4aR,7aS)-3,4a-difluoro-7a-(2-fluorophenyl)-2-oxohexahydrofuro[3,4-b]pyridine-1(2H)-carboxylate (4.24 g, 11.4 mmol) in THF (100 mL) at −78° C. The reaction mixture was stirred at −78° C. for 2 hours. Methyl iodide (3.7 mL, 60 mmol) was added. The reaction mixture was stirred at room temperature for 2 hours and was quenched with saturated ammonium chloride (aq). The mixture was extracted with ethyl acetate. The organic phase was washed with brine, dried over magnesium sulfate and concentrated in vacuo. The crude product was purified by flash chromatography on silica gel (eluent: heptane/ethyl acetate) to give I-13: tert-butyl (3R,4aR,7aS)-3,4a-difluoro-7a-(2-fluorophenyl)-3-methyl-2-oxohexahydrofuro[3,4-b]pyridine-1(2H)-carboxylate (1.86 g, 42% yield). ¹H NMR (600 MHz, CDCl₃) δ 7.44 (td, J=8.0, 1.6 Hz, 1H), 7.37 (dddd, J=7.4, 6.8, 4.9, 1.6 Hz, 1H), 7.23-7.18 (m, 1H), 7.10 (ddd, J=12.3, 8.2, 1.2 Hz, 1H), 5.08 (d, J=10.9 Hz, 1H), 4.26-4.16 (m, 2H), 4.09 (ddd, J=21.1, 10.6, 1.8 Hz, 1H), 2.79-2.61 (m, 2H), 1.78 (dd, J=23.0, 0.9 Hz, 3H), 1.23 (s, 9H).

and I-14: tert-butyl (3S,4aR,7aS)-3,4a-difluoro-7a-(2-fluorophenyl)-3-methyl-2-oxohexahydrofuro[3,4-b]pyridine-1(2H)-carboxylate (0.95 g, 22% yield). ¹H NMR (600 MHz, CDCl₃) δ 7.46 (td, J=8.0, 1.6 Hz, 1H), 7.38-7.33 (m, 1H), 7.21-7.17 (m, 1H), 7.10 (ddd, J=12.3, 8.2, 1.2 Hz, 1H), 5.06 (dd, J=10.7, 0.9 Hz, 1H), 4.22 (dd, J=10.8, 0.9 Hz, 1H), 4.07 (dd, J=16.3, 9.8 Hz, 1H), 3.96 (t, J=9.7 Hz, 1H), 2.90-2.81 (m, 1H), 2.53 (ddd, J=33.4, 31.3, 15.6 Hz, 1H), 1.73 (d, J=22.1 Hz, 3H), 1.28 (s, J=6.8 Hz, 9H).

### Intermediate I-15: (3R,4aR,7aS)-3,4a-difluoro-7a-(2-fluoro-5-nitrophenyl)-3-methylhexahydrofuro[3,4-b]pyridin-2(1H)-one

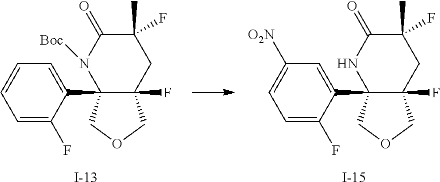

I-13: tert-butyl (3R,4aR,7aS)-3,4a-difluoro-7a-(2-fluorophenyl)-3-methyl-2-oxohexahydrofuro[3,4-b]pyridine-1(2H)-carboxylate (1.86 g, 4.83 mmol) was suspended in trifluoroacetic acid (TFA) (9.3 mL). The mixture was cooled to 0° C. and concentrated sulfuric acid (2.0 mL, 37 mmol, 97%) was added. The reaction mixture was stirred for 5 minutes at 0° C. Nitric acid (0.37 mL, 5.3 mmol, 65% in water) was added in a dropwise manner. The reaction mixture was stirred for 30 minutes at 0° C. Nitric acid (0.37 mL, 5.3 mmol, 65% in water) was added in a dropwise manner. The reaction mixture was stirred for 20 minutes at room temperature. Nitric acid (0.37 mL, 5.3 mmol, 65% in water) was added in a dropwise manner. The reaction mixture was stirred for 60 minutes at room temperature, poured onto ice and basified to pH>11 using 5 M NaOH (aq). The mixture was extracted with ethyl acetate. The organic phase was washed with brine, dried over magnesium sulfate and concentrated in vacuo to give (3R,4aR,7aS)-3,4a-difluoro-7a-(2-fluoro-5-nitrophenyl)-3-methylhexahydrofuro[3,4-b]pyridin-2(1H)-one (1.6 g, quantitative). Used in the next step without further purification. ¹H NMR (600 MHz, CDCl₃) δ 8.40 (dd, J=6.6, 2.8 Hz, 1H), 8.32 (ddd, J=8.9, 4.0, 2.8 Hz, 1H), 7.33 (dd, J=11.0, 9.0 Hz, 1H), 6.77 (bs, 1H), 4.74 (dd, J=10.0, 1.4 Hz, 1H), 4.46 (dd, J=27.3, 11.0 Hz, 1H), 4.24 (dd, J=25.7, 11.2 Hz, 1H), 4.15 (dd, J=10.0, 1.5 Hz, 1H), 2.78-2.69 (m, 1H), 2.54 (ddd, J=28.6, 15.3, 13.1 Hz, 1H), 1.63 (d, J=23.0 Hz, 3H), 1.59 (d, J=7.3 Hz, 9H).

### Intermediate I-16: (3S,4aR,7aS)-3,4a-difluoro-7a-(2-fluoro-5-nitrophenyl)-3-methylhexahydrofuro[3,4-b]pyridin-2(1H)-one

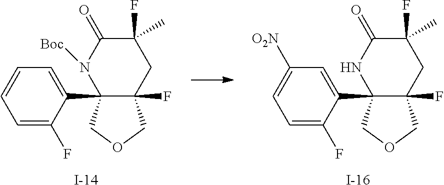

I-16: (3S,4aR,7aS)-3,4a-difluoro-7a-(2-fluoro-5-nitrophenyl)-3-methylhexahydrofuro[3,4-b]pyridin-2(1H)-one was prepared analogously to I-15 starting from I-14. ¹H NMR (600 MHz, CDCl₃) δ 8.43 (ddd, J=6.6, 2.7, 1.7 Hz, 1H), 8.28 (ddd, J=8.9, 4.1, 2.8 Hz, 1H), 7.31-7.27 (m, 1H), 6.55 (bs, 1H), 4.53 (d, J=10.3 Hz, 1H), 4.31-4.24 (m, 1H), 4.19-4.10 (m, 2H), 2.91 (ddd, J=20.8, 15.7, 12.7 Hz, 1H), 2.42 (ddd, J=33.6, 24.1, 15.7 Hz, 1H), 1.62 (d, J=22.9 Hz, 3H), 1.59 (s, 9H).

### Intermediate I-18a: tert-butyl (3-((3R,4aR,7aS)-3,4a-difluoro-3-methyl-2-oxohexahydrofuro[3,4-b]pyridin-7a-yl)-4-fluorophenyl)carbamate

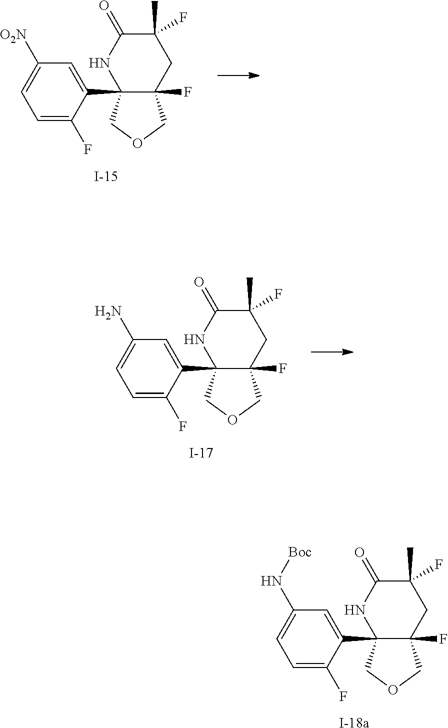

Potassium carbonate (2.52 g, 18.26 mmol) and sodium dithionite (6.36 g, 36.5 mmol) were dissolved in water (33 mL). The solution was cooled on ice/water bath. I-15 (3R,4aR,7aS)-3,4a-difluoro-7a-(2-fluoro-5-nitrophenyl)-3-methylhexahydrofuro[3,4-b]pyridin-2(1H)-one (2.01 g, 6.09 mmol) in ethanol (32.0 mL) was added dropwise maintaining the temp between 10-15° C. The mixture was allowed to warm to room temperature and stirred for 1 hour. The reaction mixture was concentrated in vacuo. Ethyl acetate and THF were added. The mixture was dried over sodium sulfate, filtered and concentrated in vacuo to give I-17. I-17 was dissolved in THF (40 mL). Di-tert-butyl dicarbonate (2.04 g, 9.32 mmol) was added. The reaction mixture was stirred at room temperature for 3 days. Di-tert-butyl dicarbonate (1.4 g, 6.4 mmol) was added.

The reaction mixture was stirred at 50° C. for 90 minutes. Di-tert-butyl dicarbonate (1.4 g, 6.4 mmol) was added. The reaction mixture was stirred at 45° C. overnight. The reaction mixture was concentrated in vacuo. A 1:1 mixture of brine/water was added. The mixture was extracted with ethyl acetate. The organic phase was washed with brine, dried over magnesium sulfate and concentrated in vacuo. The crude product was purified by flash chromatography on silica gel (eluent: heptane/ethyl acetate) to give I-18: tert-butyl (3-((3R,4aR,7aS)-3,4a-difluoro-3-methyl-2-oxohexahydrofuro[3,4-b]pyridin-7a-yl)-4-fluorophenyl)carbamate (1.45 g, 54% yield). ¹H NMR (600 MHz, CDCl₃) δ 7.46-7.35 (m, 2H), 7.05 (dd, J=11.8, 8.8 Hz, 1H), 6.72 (bs, 1H), 6.35 (bs, 1H), 4.73 (dd, J=9.8, 1.6 Hz, 1H), 4.46 (dd, J=28.4, 11.0 Hz, 1H), 4.21 (dd, J=26.8, 11.1 Hz, 1H), 4.10 (dd, J=9.9, 1.4 Hz, 1H), 2.70-2.50 (m, 2H), 1.67 (d, J=23.0 Hz, 3H), 1.51 (s, 9H).

### Intermediate I-18b: tert-butyl (3-((3S,4aR,7aS)-3,4a-difluoro-3-methyl-2-oxohexahydrofuro[3,4-b]pyridin-7a-yl)-4-fluorophenyl)carbamate

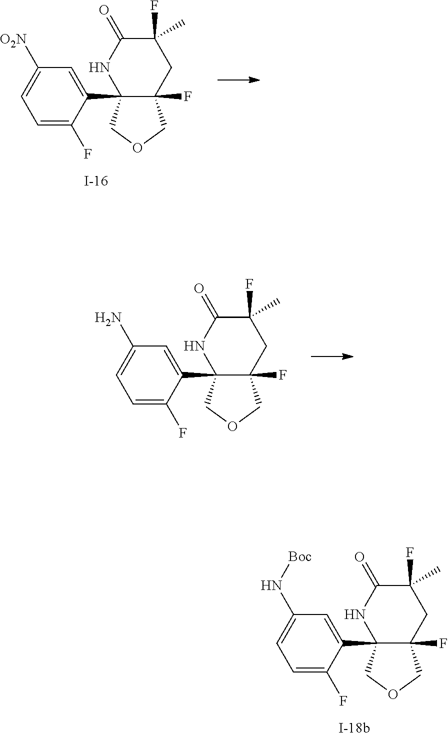

I-18b: tert-butyl (3-((3S,4aR,7aS)-3,4a-difluoro-3-methyl-2-oxohexahydrofuro[3,4-b]pyridin-7a-yl)-4-fluorophenyl)carbamate was prepared analogously to I-18a starting from I-16. ¹H NMR (600 MHz, CDCl₃) δ 7.57 (s, 1H), 7.19 (dd, J=6.6, 2.1 Hz, 1H), 7.05 (dt, J=8.6, 6.5 Hz, 1H), 6.58 (s, 1H), 6.13 (d, J=2.9 Hz, 1H), 4.58 (d, J=10.1 Hz, 1H), 4.23 (ddd, J=20.1, 10.1, 2.2 Hz, 1H), 4.16-4.10 (m, 1H), 4.02 (dd, J=10.0, 3.6 Hz, 1H), 2.86 (ddd, J=18.9, 15.2, 13.5 Hz, 1H), 2.40 (ddd, J=28.7, 23.2, 15.3 Hz, 1H), 1.64 (d, J=22.9 Hz, 3H), 1.50 (s, J=3.4 Hz, 9H).

### Intermediate I-20b: (3S,4aR,7aS)-7a-(5-amino-2-fluorophenyl)-3,4a-difluoro-3-methylhexahydrofuro[3,4-b]pyridine-2(1H)-thione

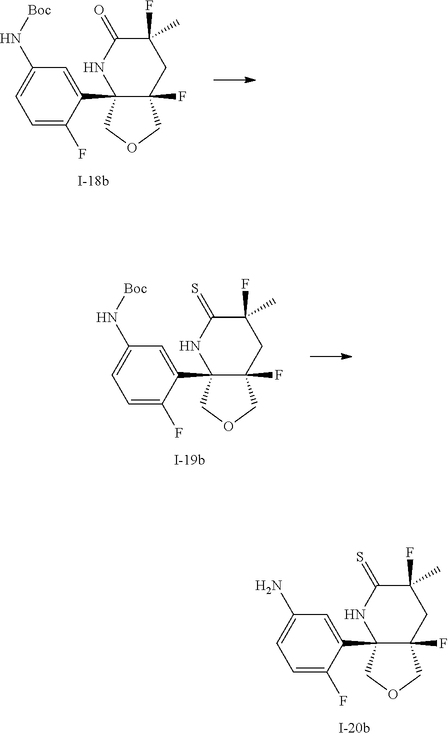

Lawesson's reagent (505 mg, 1.25 mmol) was added to a solution I-18b tert-butyl (3-((3S,4aR,7aS)-3,4a-difluoro-3-methyl-2-oxohexahydrofuro[3,4-b]pyridin-7a-yl)-4-fluorophenyl)carbamate (500 mg, 1.25 mmol) in toluene (40 mL) was stirred at 70° C. for 4 hours then overnight at room temperature. Lawesson's reagent (50 mg, 0.13 mmol) was added and the reaction mixture was stirred at 70° C. for 3 hours. The mixture was concentrated. The residue was purified by flash silica gel chromatography (eluent: ethyl acetate/heptane) to give I-19b (497 mg). Trifluoroacetic acid (5 mL) was added to I-19b (497 mg, 1.193 mmol) in dichloromethane (5 mL). The reaction mixture was stirred at room temperature for 30 minutes. The mixture was basified with 2M NaOH (aq) and extracted with ethyl acetate. The organic phase was washed with brine, dried over magnesium sulfate and concentrated in vacuo. The crude product was purified by flash chromatography on silica gel (eluent: heptane/ethyl acetate) to give I-20b (3S,4aR,7aS)-7a-(5-amino-2-fluorophenyl)-3,4a-difluoro-3-methylhexahydrofuro[3,4-b]pyridine-2(1H)-thione (203 mg, 54%). LC-MS (m/z) 317.1 (MH⁺) t_{R}=0.38 minutes (Method A). I-20a (3R,4aR,7aS)-7a-(5-amino-2-fluorophenyl)-3,4a-difluoro-3-methylhexahydrofuro[3,4-b]pyridine-2(1H)-thione was prepared in a similar way from I-18a tert-butyl (3-((3S,4aR,7aS)-3,4a-difluoro-3-methyl-2-oxohexahydrofuro[3,4-b]pyridin-7a-yl)-4-fluorophenyl)carbamate.

### Intermediate I-21: tert-butyl ((3S,4R)-3-(2,3-difluorophenyl)-4-(hydroxymethyl)tetrahydrofuran-3-yl)carbamate

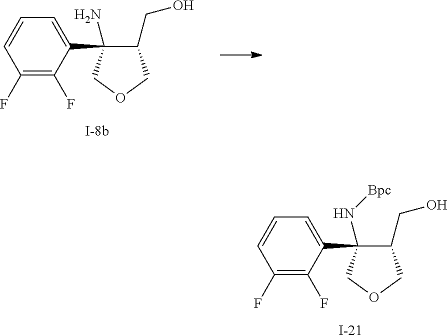

A solution of I-8b ((3R,4S)-4-amino-4-(2,3-difluorophenyl)tetrahydrofuran-3-yl)methanol (11.93 g, crude) and Boc₂O (12.50 g, 57.26 mmol) in THF (45 mL) was stirred at 60° C. for 16 h. The mixture was concentrated. The residue was purified by flash silica gel chromatography (eluent of 0-50% ethyl acetate/petroleum ether) to give tert-butyl ((3S,4R)-3-(2,3-difluorophenyl)-4-(hydroxymethyl)tetrahydrofuran-3-yl)carbamate (15.0 g, 45.6 mmol, 88% yield over two steps). LC-MS (m/z) 274.0 (MH⁺-t-Bu) t_{R}=0.65 minutes (Method B).

### Intermediate I-22: (3S,4S)-4-((tert-butoxycarbonyl)amino)-4-(2,3-difluorophenyl)tetrahydrofuran-3-carboxylic acid

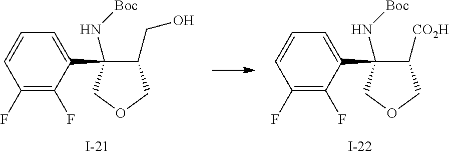

A mixture of tert-butyl ((3S,4R)-3-(2,3-difluorophenyl)-4-(hydroxymethyl)tetrahydrofuran-3-yl)carbamate (26.00 g, 78.95 mmol), NalO₄ (67.54 g, 315.78 mmol) and RuCl₃ (164 mg, 0.789 mmol) in CCl₄ (250 mL), MeCN (250 mL) and water (375 mL) was stirred at 15° C. for 3 h. The mixture was diluted with EtOAc (500 mL) and washed with brine (3×200 mL), dried over Na₂SO₄, filtered and concentrated to give (3S,4S)-4-((tert-butoxycarbonyl)amino)-4-(2,3-difluorophenyl)tetrahydrofuran-3-carboxylic acid (26.31 g, crude) which was used for next step directly without further purification. LC-MS (m/z) 278.1 (M-H⁺) t_{R}=0. 745 minutes (Method E).

### Intermediate I-22: tert-butyl ((3S,4S)-3-(2,3-difluorophenyl)-4-(2,2-dimethyl-4,6-dioxo-1,3-dioxane-5-carbonyl)tetrahydrofuran-3-yl)carbamate

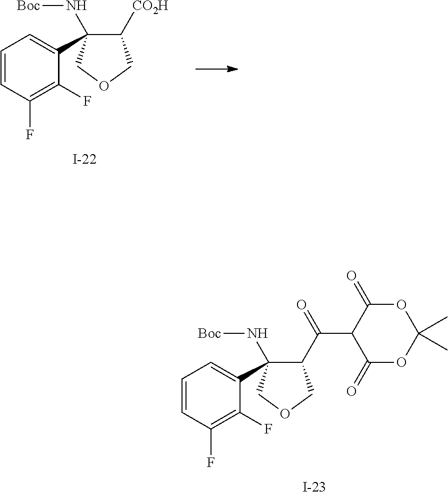

To a solution of crude (3S,4S)-4-((tert-butoxycarbonyl)amino)-4-(2,3-difluorophenyl)tetrahydrofuran-3-carboxylic acid (26 g), DMAP (13.88 g, 113.60 mmol) and EDC (21.78 g, 113.6 mmol) in THF (1.25 L) was added (3S,4S)-4-((tert-butoxycarbonyl)amino)-4-(2,3-difluorophenyl)tetrahydrofuran-3-carboxylic acid (14.19 g, 98.45 mmol) at 10° C. The mixture was stirred at 10° C. for 5 h. The mixture was filtered and concentrated. The residue was dissolved in dichloromethane (600 mL). The solution was washed with water (2×300 mL) and brine (300 mL), dried over Na₂SO₄, filtered and concentrated to give crude tert-butyl ((3S,4S)-3-(2,3-difluorophenyl)-4-(2,2-dimethyl-4,6-dioxo-1,3-dioxane-5-carbonyl)tetrahydrofuran-3-yl)carbamate (36 g) which was used for next step directly without further purification. LC-MS (m/z) 468.1 (M-H⁺) t_{R}=1.206 minutes (Method E)

### Intermediate I-24: tert-butyl ((3S,4S)-3-(2,3-difluorophenyl)-4-((2,2-dimethyl-4,6-dioxo-1,3-dioxan-5-yl)methyl)tetrahydrofuran-3-yl)carbamate

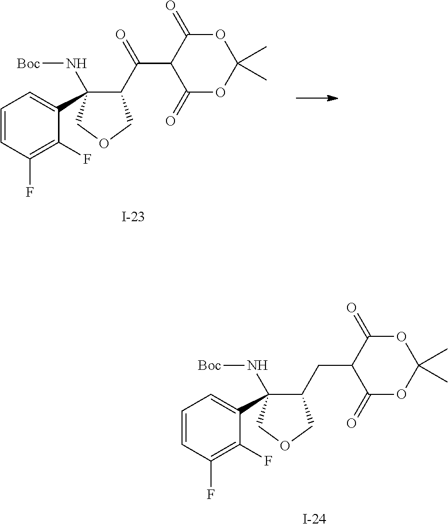

To a solution of crude tert-butyl ((3S,4S)-3-(2,3-difluorophenyl)-4-(2,2-dimethyl-4,6-dioxo-1,3-dioxane-5-carbonyl)tetrahydrofuran-3-yl)carbamate (36 g) in AcOH (200 mL) was added NaBH₄ (14.49 g, 383.12 mmol) in 6 portions during 2 h at 15° C. The mixture was stirred at 15° C. for 8 h. The reaction was quenched with water (800 mL) and extracted with dichloromethane (3×500 mL). The combined organic layers were washed with brine (1000 mL), dried over Na₂SO₄, filtered and concentrated to give crude tert-butyl ((3S,4S)-3-(2,3-difluorophenyl)-4-((2,2-dimethyl-4,6-dioxo-1,3-dioxan-5-yl)methyl)tetrahydrofuran-3-yl)carbamate (34.90 g) which was used for next step directly without further purification. LC-MS (m/z) 454.2 (M-H⁺) t_{R}=1.08 minutes (Method E).

### Intermediate I-25: methyl (4aS,7aS)-7a-(2,3-difluorophenyl)-2-oxooctahydrofuro[3,4-b]pyridine-3-carboxylate

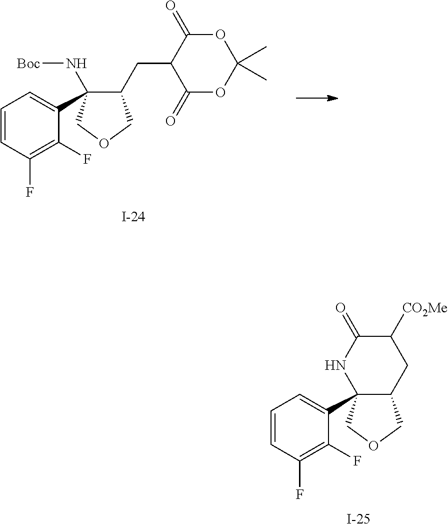

A solution of crude tert-butyl ((3S,4S)-3-(2,3-difluorophenyl)-4-((2,2-dimethyl-4,6-dioxo-1,3-dioxan-5-yl)methyl)tetrahydrofuran-3-yl)carbamate (34.90 g) in HCl/MeOH (250 mL, 4.0 M) and MeOH (250 mL) was stirred at 15° C. for 3 h. The mixture was concentrated in vacuo. The residue was dissolved and in MeOH (500 mL) and Et₃N (38.8 g, 383 mmol) and was stirred at 65° C. for 16 hours under Ar. The mixture was concentrated. To the residue was added dichloromethane (200 mL). The mixture was filtered and concentrated. The residue was purified by flash silica gel chromatography (eluent of 0100% ethyl acetate/petroleum ether) to give methyl (4aS,7aS)-7a-(2,3-difluorophenyl)-2-oxooctahydrofuro[3,4-b]pyridine-3-carboxylate (15.27 g, 49.06 mmol). LC-MS (m/z) 312.1 (MH⁺) t_{R}=0.648 minutes (Method F)

### Intermediate I-26: methyl (4aS,7aS)-7a-(2,3-difluorophenyl)-3-fluoro-2-oxooctahydrofuro[3,4-b]pyridine-3-carboxylate

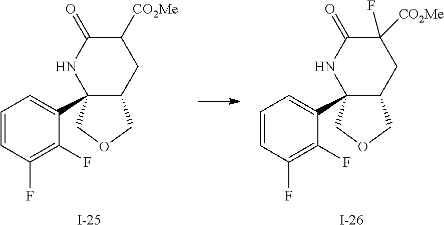

To a solution of methyl (4aS,7aS)-7a-(2,3-difluorophenyl)-2-oxooctahydrofuro[3,4-b]pyridine-3-carboxylate (3.00 g, 9.64 mmol in THF (40 mL) was added NFSi (4.56 g, 14.46 mmol) and DBU (2.20 g, 14.46 mmol) at 15° C. The reaction was stirred at 15° C. for 16 hours. The mixture was filtered and concentrated to give crude methyl (4aS,7aS)-7a-(2,3-difluorophenyl)-3-fluoro-2-oxooctahydrofuro[3,4-b]pyridine-3-carboxylate (6.00 g) which was used for next step directly without further purification. LC-MS (m/z) 328.0 (M-H⁺) t_{R}=1.516 minutes (Method E).

### Intermediate I-27: (4aS,7aS)-7a-(2,3-difluorophenyl)-3-fluoro-3-(hydroxymethyl)hexahydrofuro[3,4-b]pyridin-2(1H)-one

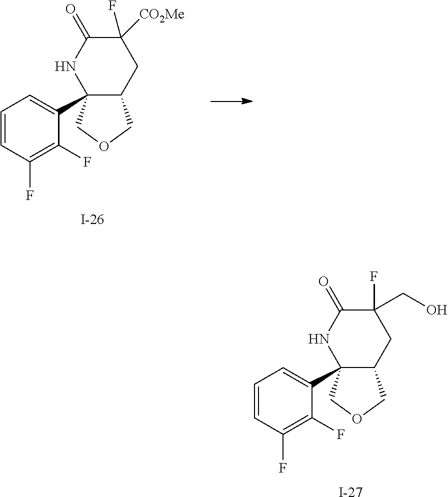

To a solution of crude methyl (4aS,7aS)-7a-(2,3-difluorophenyl)-3-fluoro-2-oxooctahydrofuro[3,4-b]pyridine-3-carboxylate (6.00 g) in MeOH (100 mL) was added NaBH₄ (6.89 g, 182.20 mmol) in portions at 20° C. The reaction was stirred at 20° C. for 20 hours. Additional NaBH₄ (3.00 g) was added into the reaction in portions. The reaction was stirred at 20° C. for 20 hours. The mixture was concentrated. To the mixture was added water (150 mL). The mixture was extracted with ethyl acetate (3×100 mL). The combined organic layer was washed with brine (200 mL), dried over Na₂SO₄, filtered and concentrated. The residue with another batch of the same scale was purified by flash silica gel chromatography (eluent of 0-10% MeOH/dichloromethane) to give (4aS,7aS)-7a-(2,3-difluorophenyl)-3-fluoro-3-(hydroxymethyl)hexahydrofuro[3,4-b]pyridin-2(1H)-one (4.99 g, 16.6 mmol)

### Intermediate I-28: (4aS,7aS)-7a-(2,3-difluorophenyl)-3-fluoro-3-(fluoromethyl)hexahydrofuro[3,4-b]pyridin-2(1H)-one

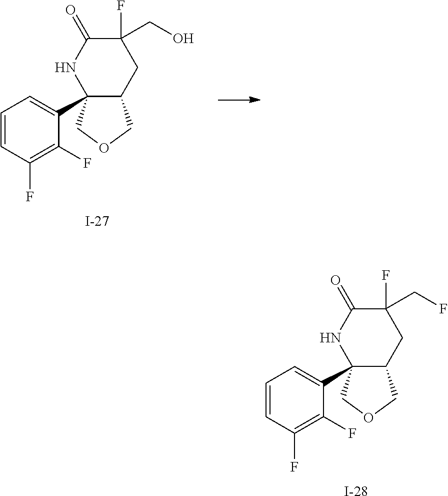

To a solution of (4aS,7aS)-7a-(2,3-difluorophenyl)-3-fluoro-3-(hydroxymethyl)hexahydrofuro[3,4-b]pyridin-2(1H)-one (4.50 g, 14.94 mmol) and NfF (1,1,2,2,3,3,4,4,4-nonafluorobutane-1-sulfonyl fluoride) (36.10 g, 119.5 mmol) in THF (100 mL) was added Et₃N (18.14 g, 179.2 mmol) at 15° C. The reaction was stirred at 15° C. for 16 hours. TBAF (1.0 M, 8.97 mL) (1.0 M in THF) was added and the reaction mixture was stirred at 50° C. for 20 hours. To the mixture was added water (150 mL). The mixture was extracted with ethyl acetate (4×100 mL). The combined organic layer was washed with brine (300 mL), dried over Na₂SO₄, filtered and concentrated. The residue was purified by flash silica gel chromatography (eluent of 0˜50% ethyl acetate/petroleum ether) to give (4aS,7aS)-7a-(2,3-difluorophenyl)-3-fluoro-3-(fluoromethyl)hexahydrofuro[3,4-b]pyridin-2(1H)-one (2.46 g, 8.11 mmol, 54% yield) LC-MS (m/z) 304.1 (MH⁺) t_{R}=0.657-0.706 minutes (Method F)

### Intermediate I-29: (4aS,7aS)-7a-(2,3-difluoro-5-nitrophenyl)-3-fluoro-3-(fluoromethyl)hexahydrofuro[3,4-b]pyridin-2(1H)-one

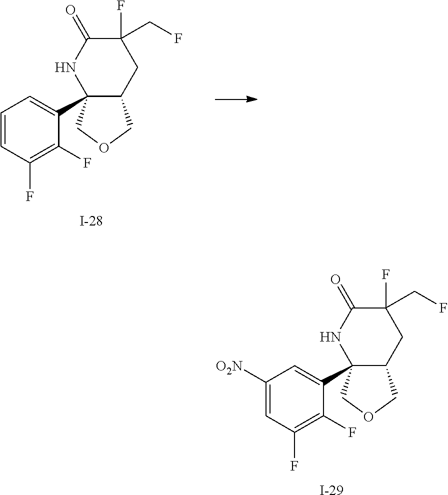

To a solution of (4aS,7aS)-7a-(2,3-difluorophenyl)-3-fluoro-3-(fluoromethyl)hexahydrofuro[3,4-b]pyridin-2(1H)-one (2.50 g, 8.24 mmol) in TFA (20.68 g, 181.4 mmol) was added H₂SO₄ (6.23 g, 63.5 mmol) and HNO₃ (60%, 2.60 g, 24.7 mmol) dropwise at 0° C. The mixture was stirred at 0° C. for 2 hours, then warmed to 15° C. and stirred for 20 hours. The reaction solution was poured into crushed ice (300 mL), and for the above mixture, pH was adjusted to 11 with NaOH (5% aq). The mixture was extracted with ethyl acetate (2×300 mL). The combined organic layers were washed with brine (100 mL), dried over Na₂SO₄, filtered and concentrated to give crude (4aS,7aS)-7a-(2,3-difluoro-5-nitrophenyl)-3-fluoro-3-(fluoromethyl)hexahydrofuro[3,4-b]pyridin-2(1H)-one (2.50 g).

### Intermediate I-31: (4aS,7aS)-7a-(5-amino-2,3-difluorophenyl)-3-fluoro-3-(fluoromethyl)hexahydrofuro[3,4-b]pyridine-2(1H)-thione

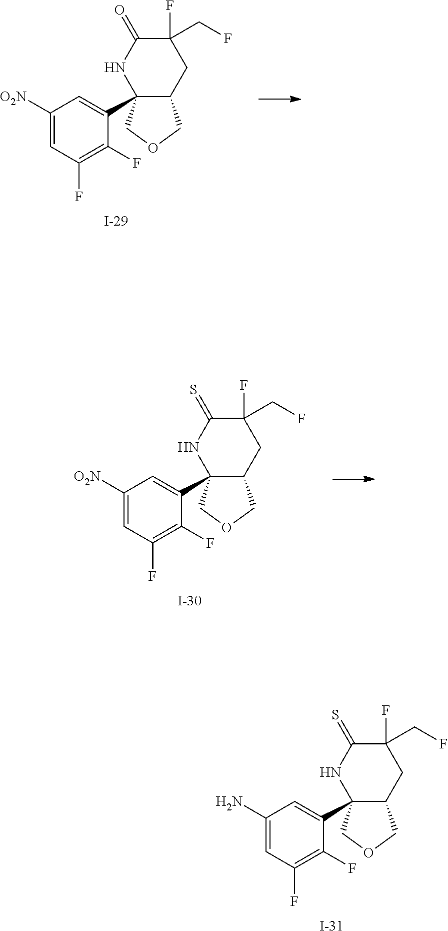

A solution of crude (4aS,7aS)-7a-(2,3-difluoro-5-nitrophenyl)-3-fluoro-3-(fluoromethyl)hexahydrofuro[3,4-b]pyridin-2(1H)-one (2.50 g) and Lawesson's reagent (1.71 g, 4.23 mmol) in toluene (40 mL) was stirred at 110° C. for 2 hours under N₂. The mixture was concentrated. The residue was purified by flash silica gel chromatography (eluent of 0-30% ethyl acetate/petroleum ether) to give I-30 (2.6 g). A mixture of I-30 (2.6 g), Fe powder (1.99 g, 35.6 mmol) and NH₄Cl (1.90 g, 35.6 mmol) in EtOH (40 mL) and water (10 mL) was stirred at 25° C. for 4 hours. The mixture was filtered over a layer of celite. The filtrate was concentrated. To the residue was added dichloromethane (100 mL). The mixture was filtered and the filtrate was concentrated. The residue was purified by flash silica gel chromatography (eluent of 0-50% ethyl acetate/petroleum ether) to give (4aS,7aS)-7a-(5-amino-2,3-difluorophenyl)-3-fluoro-3-(fluoromethyl)hexahydrofuro[3,4-b]pyridine-2(1H)-thione (1.80 g, 5.38 mmol). LC-MS (m/z) 335.1 (MH⁺) t_{R}=0.689 minutes, 0.703 minutes (Method F)

### Intermediates I-32a and I-32b: (3R,4aS,7aS)-7a-(5-amino-2,3-difluorophenyl)-3-fluoro-3-(fluoromethyl)hexahydrofuro[3,4-b]pyridine-2(1H)-thione and (3S,4aS,7aS)-7a-(5-amino-2,3-difluorophenyl)-3-fluoro-3-(fluoromethyl)hexahydrofuro[3,4-b]pyridine-2(1H)-thione

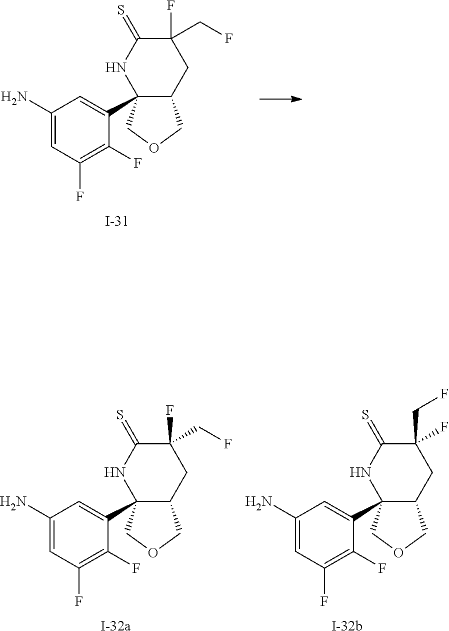

I-31 (4aS,7aS)-7a-(5-amino-2,3-difluorophenyl)-3-fluoro-3-(fluoromethyl)hexahydrofuro[3,4-b]pyridine-2(1H)-thione 1.80 g, 5.38 mmol) was purified by SFC: (Instrument: SFC 9, Column: AD (250 mm×30 mm, 5 um., Mobile phase: A: Supercritical CO₂, B: EtOH (base), A:B=60:40 at 200 mL/min, Column Temp: 38° C.; Nozzle Pressure: 100 Bar, Nozzle Temp: 60° C., Evaporator Temp: 20° C., Trimmer Temp: 25° C., Wavelength: 220 nm) to give (3R,4aS,7aS)-7a-(5-amino-2,3-difluorophenyl)-3-fluoro-3-(fluoromethyl)hexahydrofuro[3,4-b]pyridine-2(1H)-thione (1.03 g, 3.08 mmol, 57% yield) and (3S,4aS,7aS)-7a-(5-amino-2,3-difluorophenyl)-3-fluoro-3-(fluoromethyl)hexahydrofuro[3,4-b]pyridine-2(1H)-thione (520 mg, 1.56 mmol, 29% yield).

I-32a (3R,4aS,7aS)-7a-(5-amino-2,3-difluorophenyl)-3-fluoro-3-(fluoromethyl)hexahydrofuro[3,4-b]pyridine-2(1H)-thione ¹H NMR (CDCl₃ 400 MHz): δ 8.23 (s, 1H), 6.48-6.45 (m, 1H), 6.32 (t, J=2.6 Hz, 1H), 5.15-5.02 (m, 1H), 4.80-4.61 (m, 1H), 4.22 (t, J=7.2 Hz, 1H), 4.12 (d, J=3.2 Hz, 2H), 3.93 (d, J=9.2 Hz, 1H), 3.73 (s, 2H), 3.2-3.19 (m, 1H), 2.75-2.71 (m, 1H), 2.02-1.18 (m, 1H).

I-32b (3S,4aS,7aS)-7a-(5-amino-2,3-difluorophenyl)-3-fluoro-3-(fluoromethyl)hexahydrofuro[3,4-b]pyridine-2(1H)-thione ¹H NMR (CDCl₃ 400 MHz): δ 8.26 (s, 1H), 6.52-6.48 (m, 1H), 6.28 (t, J=2.6 Hz, 1H), 4.91-4.76 (m, 1H), 4.66-4.46 (m, 1H), 4.29-4.21 (m, 2H), 4.12 (dd, J=9.6, 7.6 Hz, 2H), 4.06 (t, J=7.6 Hz, 2H), 3.79 (s, 2H), 3.19-3.13 (m, 1H), 2.44-2.33 (m, 2H).

(3R,4aS,7aS)-7a-(5-amino-2-fluorophenyl)-3-fluoro-3-(fluoromethyl)hexahydrofuro[3,4-b]pyridine-2(1H)-thione and (3S,4aS,7aS)-7a-(5-amino-2-fluorophenyl)-3-fluoro-3-(fluoromethyl)hexahydrofuro[3,4-b]pyridine-2(1H)-thione were prepared in a similar way starting with ((3R,4S)-4-amino-4-(2-fluorophenyl)tetrahydrofuran-3-yl)methanol as intermediate I-8a.

### Intermediate: methyl 5-(methoxy-d₃)picolinate

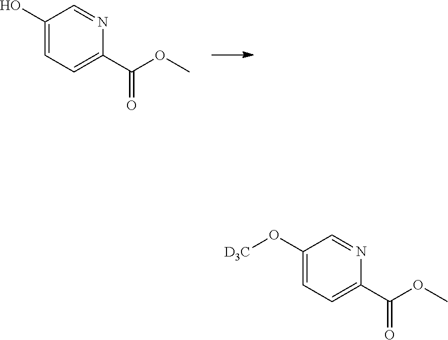

Methyl 5-hydroxypicolinate (2.88 g, 18.8 mmol) was dissolved in dimethylformamide (108 ml) under argon. Potassium carbonate (7.20 g, 52.1 mmol) was added and the orange suspension was stirred for 45 minutes at room temperature. Iodomethane-d₃ (1.41 ml, 22.6 mmol) was added. The reaction mixture was stirred for 2 hours. Water was added. The mixture was extracted with ethyl acetate. The organic phase was washed with brine, dried over MgSO₄ and concentrated in vacuo and purified by column chromatography on silica gel (heptane: ethyl acetate) to give methyl 5-(methoxy-d₃)picolinate. LC-MS (m/z) 171.1 (MH⁺); t_{R}=0.35 (Method B)

### Intermediate: 5-(methoxy-d₃)picolinic acid

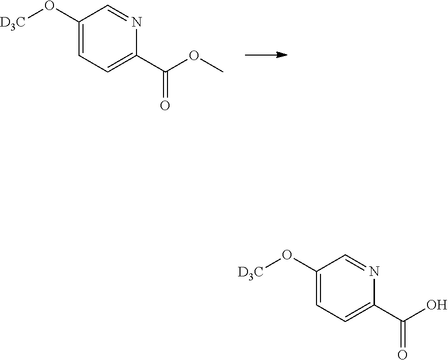

Methyl 5-(methoxy-d₃)picolinate (200 mg, 1.175 mmol) was dissolved in water (1.5 ml) and 1,4-dioxane (3 ml). Lithium hydroxide (70.4 mg, 2.94 mmol) was added and the reaction mixture was stirred for 1 hour. The reaction mixture was evaporated to about 2 ml and extracted with diethyl ether. The organic phase was extracted with 1M NaOH and the combined aqueous phases were acidified to pH 2 with 6N HCl (aq). The mixture was cooled on an ice bath and a precipitate was formed. The precipitate was collected to give 5-(methoxy-d₃)picolinic acid. LC-MS (m/z) 157.0 (MH⁺); t_{R}=0.20 (Method B)

### Intermediate: methyl-d₃-5-(methoxy-d₃)pyrazine-2-carboxylate

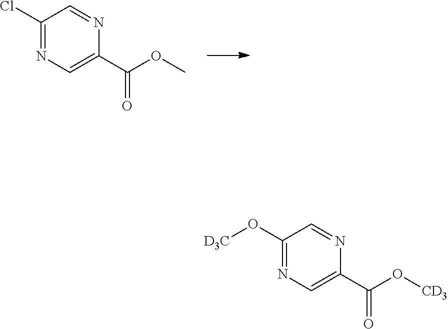 [SMILES: COC(=O)c1cnc(Cl)cn1 ; [2H]C([2H])([2H])OC(=O)c1cnc(OC([2H])([2H])[2H])cn1]

Sodium (0.094 g, 4.10 mmol) was added in small portions methanol-d₄ (2.94 ml) and the reaction mixture was stirred until all sodium has reacted. The solution was the added to another solution of methyl-5-chloropyrazine-2-carboxylate (0.6 g, 3.48 mmol) in methanol-d₄ (0.98 ml). The reaction mixture was stirred for 1.5 hours at room temperature. The reaction mixture was concentrated in vacuo. 2 ml of water was added. The mixture was extracted with ethyl acetate.

The organic phase was washed with brine, dried over MgSO₄ and concentrated in vacuo to give methyl-d₃-5-(methoxy-d₃)pyrazine-2-carboxylate. LC-MS (m/z) 175.1 (MH⁺); t_{R}=0.49 (Method A)

### Intermediate: 5-(methoxy-d₃)pyrazine-2-carboxylic acid

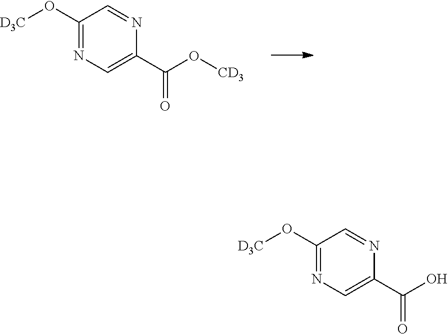 [SMILES: [2H]C([2H])([2H])OC(=O)c1cnc(OC([2H])([2H])[2H])cn1 ; [2H]C([2H])([2H])Oc1cnc(C(=O)O)cn1]

Methyl-d₃-5-(methoxy-d₃)pyrazine-2-carboxylate (424 mg, 2.43 mmol) was dissolved in water (3 ml) and 1,4-dioxane (3 ml). Lithium hydroxide (146 mg, 6.09 mmol) was added and the reaction mixture was stirred for 1 hour. The reaction mixture was evaporated to about 2 ml and extracted with diethyl ether. The organic phase was extracted with 1M NaOH and the combined aqueous phases were acidified to pH 2 with 6N HCl (aq). The mixture was cooled on an ice bath, and the solid compound collected to give 5-(methoxy-d₃)pyrazine-2-carboxylic acid. LC-MS (m/z) 158.1 (MH⁺); t_{R}=0.27 (Method A)

### Stereochemistry

The relative stereochemistries of intermediates I-32a (3R,4aS,7aS)-7a-(5-amino-2,3-difluorophenyl)-3-fluoro-3-(fluoromethyl)hexahydrofuro[3,4-b]pyridine-2(1H)-thione and I-32b (3S,4aS,7aS)-7a-(5-amino-2,3-difluorophenyl)-3-fluoro-3-(fluoromethyl)hexahydrofuro[3,4-b]pyridine-2(1H)-thione were assigned by two-dimensional ¹H-¹⁹F HOESY (heteronuclear nuclear Overhauser effect spectroscopy) (FIG. 1). For I-32a, nOe (nuclear Overhauser effect) signals were observed between F(A) (δ −154) and H(B) (δ 3.2-3.19) and between F(A) (δ −154) and H(D) (δ 6.32). For I-32b, nOe (nuclear Overhauser effect) signals were observed between F(E) (δ −231) and H(B) (δ 3.19-3.13) and between F(E) (δ −231) and H(D) (δ 6.28) and an nOe signal was also observed between F(A) (δ −150) and H(C) (δ 4.06).

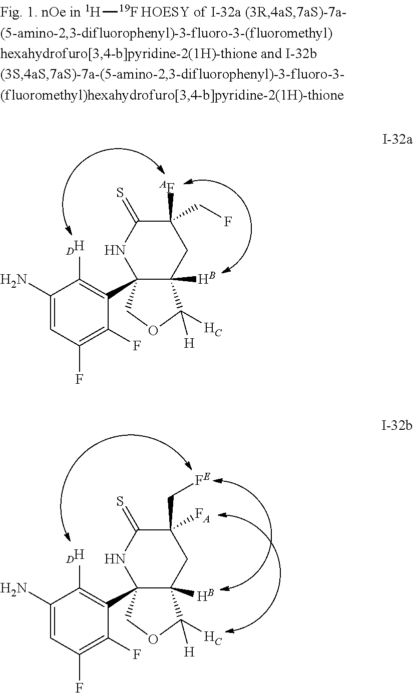

The relative stereochemistries of intermediates (3R,4aS,7aS)-7a-(5-amino-2-fluorophenyl)-3-fluoro-3-(fluoromethyl)hexahydrofuro[3,4-b]pyridine-2(1H)-thione and (3S,4aS,7aS)-7a-(5-amino-2-fluorophenyl)-3-fluoro-3-(fluoromethyl)hexahydrofuro[3,4-b]pyridine-2(1H)-thione were assigned by analogy.

The relative stereochemistry of intermediate I-20a (3R,4aR,7aS)-7a-(5-amino-2-fluorophenyl)-3,4a-difluoro-3-methylhexahydrofuro[3,4-b]pyridine-2(1H)-thione was assigned by 2D ROESY (rotating frame nuclear Overhauser effect spectroscopy) (FIG. 2). nOe signals were observed between H(A) (δ 1.63) and H(B) (δ 2.44), between H(A) (δ 1.63) and H(C) (δ 6.49) and between H(B) (δ 2.44) and H(C) (δ 6.49). Thus the methyl group and the phenyl ring must be on the same side of the thiolactam ring. By consequence thereof, the relative stereochemistry of intermediate I-20b: (3S,4aR,7aS)-7a-(5-amino-2-fluorophenyl)-3,4a-difluoro-3-methylhexahydrofuro[3,4-b]pyridine-2(1H)-thione is also confirmed.

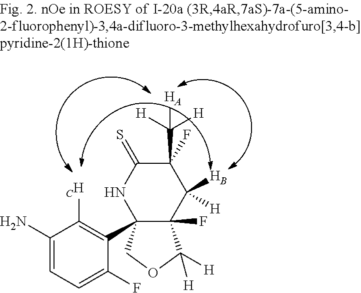

### Preparation of BACE Inhibitors

### Example 1 N-(3-((3S,4aR,7aS)-2-amino-3,4a-difluoro-3-methyl-3,4,4a,5-tetrahydrofuro[3,4-b]pyridin-7a-yl)-4-fluorophenyl)-5-(methoxy-d₃)picolinamide

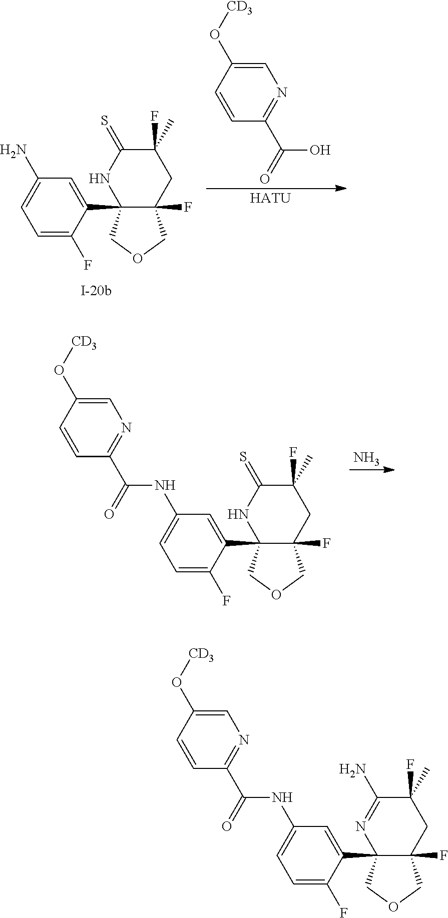

HATU (1-[Bis(dimethylamino)methylene]-1H-1,2,3-triazolo[4,5-b]pyridinium 3-oxid hexafluorophosphate) (313 mg, 0.822 mmol) was added to 5-(methoxy-d₃)picolinic acid (128 mg, 0.822 mmol) in DMF (10 ml). The reaction mixture was stirred at room temperature for 10 minutes. I-20b (3S,4aR,7aS)-7a-(5-amino-2-fluorophenyl)-3,4a-difluoro-3-methylhexahydrofuro[3,4-b]pyridine-2(1H)-thione was added followed by DIPEA (N,N-diisopropylethylamine) (0.55 mL) and the reaction mixture was stirred at room temperature for 3 days. Saturated ammonium chloride (aq) was added. The mixture was extracted with ethyl acetate. The organic phase was washed with brine, dried over magnesium sulfate and concentrated in vacuo. 7M ammonia in methanol (15 mL, 105 mmol) was added and the reaction mixture was stirred in a sealed vial at 55° C. overnight. The reaction mixture was allowed to cool to room temperature and was concentrated in vacuo. The crude product was purified by flash chromatography on silica gel (eluent: heptane/ethyl acetate). The product was further purified by the following procedure: The product was dissolved in ethyl acetate (50 mL) and washed with a solution of saturated aqueous NaHCO3/water (1/1). The organic phase was washed total of 10 times (using 10 mL each time). The organic phase was dried over MgSO4, filtered, and evaporated to give N-(3-((3S,4aR,7aS)-2-amino-3,4a-difluoro-3-methyl-3,4,4a,5-tetrahydrofuro[3,4-b]pyridin-7a-yl)-4-fluorophenyl)-5-(methoxy-d₃)picolinamide. ¹H NMR (600 MHz, CDCl₃) δ 9.84 (s, 1H), 8.27-8.25 (m, 1H), 8.25-8.23 (m, 1H), 7.85 (ddd, J=8.7, 4.0, 2.8 Hz, 1H), 7.70 (dd, J=6.7, 2.7 Hz, 1H), 7.34 (dd, J=8.7, 2.9 Hz, 1H), 7.08 (dd, J=11.6, 8.8 Hz, 1H), 4.74 (d, J=9.0 Hz, 1H), 4.22-4.09 (m, 2H), 3.97 (dd, J=9.0, 1.9 Hz, 1H), 2.67-2.57 (m, 1H), 2.41 (dd, J=12.7, 3.3 Hz, 1H), 1.82 (d, J=24.6 Hz, 3H). LC-MS (m/z) 437.438 (MH⁺); t_{R}=0.48 (Method A)

The following compounds were prepared in a way similar to the compound of Example 1:

### Example 2 N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-fluoropicolinamide

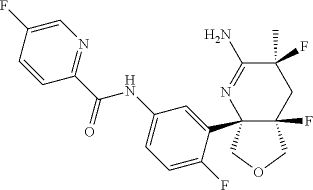 [SMILES: C[C@]1(F)C[C@]2(F)COC[C@]2(c2cc(NC(=O)c3ccc(F)cn3)ccc2F)N=C1N]

Prepared from (3S,4aR,7aS)-7a-(5-amino-2-fluorophenyl)-3,4a-difluoro-3-methylhexahydrofuro[3,4-b]pyridine-2(1H)-thione and 5-fluoropicolinic acid ¹H NMR (600 MHz, DMSO) δ 10.70 (s, 1H), 8.74 (d, J=2.9 Hz, 1H), 8.23 (dd, J=8.7, 4.6 Hz, 1H), 7.99 (td, J=8.7, 2.9 Hz, 1H), 7.91-7.88 (m, 2H), 7.12 (dd, J=11.7, 9.6 Hz, 1H), 6.09 (s, 2H), 4.50 (d, J=8.8 Hz, 1H), 4.16 (dt, J=20.0, 10.0 Hz, 1H), 3.90 (dd, J=25.4, 10.8 Hz, 1H), 3.83 (d, J=9.7 Hz, 1H), 2.58-2.51 (m, 2H), 1.68 (d, J=24.2 Hz, 3H). LC-MS (m/z) 422.37 (MH⁺); t_{R}=0.5 (Method A).

### Example 3 N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-(difluoromethyl)pyrazine-2-carboxamide

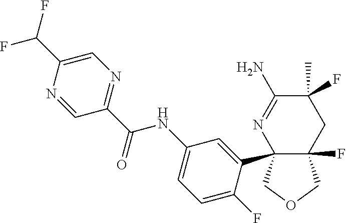 [SMILES: C[C@]1(F)C[C@]2(F)COC[C@]2(c2cc(NC(=O)c3cnc(C(F)F)cn3)ccc2F)N=C1N]

Prepared from (3S,4aR,7aS)-7a-(5-amino-2-fluorophenyl)-3,4a-difluoro-3-methylhexahydrofuro[3,4-b]pyridine-2(1H)-thione and 5-(difluoromethyl)pyrazine-2-carboxylic acid ¹H NMR (600 MHz, DMSO) δ 10.99 (s, 1H), 9.40 (d, J=1.4 Hz, 1H), 9.10 (d, J=0.9 Hz, 1H), 7.95 (dd, J=7.1, 2.6 Hz, 1H), 7.92 (ddd, J=8.7, 4.0, 2.8 Hz, 1H), 7.27 (t, J=54.0 Hz, 1H), 7.16 (dd, J=11.8, 8.8 Hz, 1H), 6.12 (s, 2H), 4.51 (d, J=8.9 Hz, 1H), 4.17 (dd, J=17.2, 10.7 Hz, 1H), 3.91 (dd, J=25.2, 10.7 Hz, 1H), 3.84 (dd, J=8.8, 1.3 Hz, 1H), 2.61-2.52 (m, 2H), 1.69 (d, J=24.2 Hz, 3H). LC-MS (m/z) 455.38 (MH⁺); t_{R}=0.47 (Method A).

### Example 4 N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-methoxypicolinamide

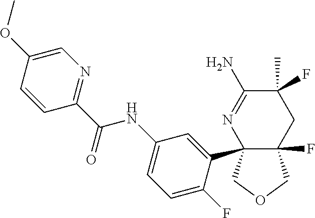 [SMILES: COc1ccc(C(=O)Nc2ccc(F)c([C@]34COC[C@@]3(F)C[C@](C)(F)C(N)=N4)c2)nc1]

Prepared from (3S,4aR,7aS)-7a-(5-amino-2-fluorophenyl)-3,4a-difluoro-3-methylhexahydrofuro[3,4-b]pyridine-2(1H)-thione and 5-methoxypicolinic acid ¹H NMR (600 MHz, DMSO) δ 10.55 (s, 1H), 8.40 (dd, J=2.9, 0.5 Hz, 1H), 8.14 (dd, J=8.7, 0.5 Hz, 1H), 7.92-7.87 (m, 2H), 7.62 (dd, J=8.8, 2.9 Hz, 1H), 7.11 (dd, J=11.7, 8.7 Hz, 1H), 6.11 (s, 2H), 4.52 (d, J=8.8 Hz, 1H), 4.17 (dd, J=17.0, 10.7 Hz, 1H), 3.94 (s, 3H), 3.95-3.87 (m, 1H), 3.84 (d, J=8.5 Hz, 1H), 2.58-2.51 (m, 2H), 1.69 (d, J=24.2 Hz, 3H). LC-MS (m/z) 434.41 (MH⁺); t_{R}=0.49 (Method A).

### Example 5 N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-methoxypyrazine-2-carboxamide

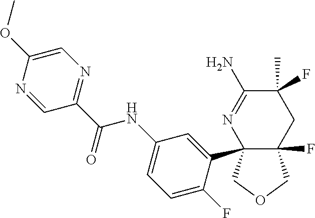 [SMILES: COc1cnc(C(=O)Nc2ccc(F)c([C@]34COC[C@@]3(F)C[C@](C)(F)C(N)=N4)c2)cn1]

Prepared from (3S,4aR,7aS)-7a-(5-amino-2-fluorophenyl)-3,4a-difluoro-3-methylhexahydrofuro[3,4-b]pyridine-2(1H)-thione and 5-methoxypyrazine-2-carboxylic acid ¹H NMR (600 MHz, DMSO) δ 10.59 (s, 1H), 8.90 (d, J=1.3 Hz, 1H), 8.42 (d, J=1.3 Hz, 1H), 7.90 (dd, J=7.1, 2.6 Hz, 1H), 7.87 (ddd, J=8.7, 4.0, 2.7 Hz, 1H), 7.12 (dd, J=11.7, 8.8 Hz, 1H), 6.08 (s, 2H), 4.50 (d, J=8.8 Hz, 1H), 4.16 (dd, J=17.1, 10.7 Hz, 1H), 4.02 (s, 3H), 3.90 (dd, J=25.4, 10.8 Hz, 1H), 3.83 (d, J=8.8 Hz, 1H), 2.57-2.50 (m, 2H), 1.68 (d, J=24.3 Hz, 3H). LC-MS (m/z) 435.4 (MH⁺); t_{R}=0.47 (Method A).

### Example 6 N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-chloropicolinamide

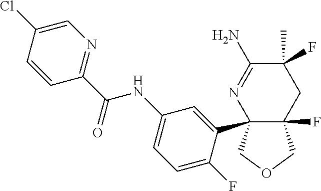 [SMILES: C[C@]1(F)C[C@]2(F)COC[C@]2(c2cc(NC(=O)c3ccc(Cl)cn3)ccc2F)N=C1N]

Prepared from (3S,4aR,7aS)-7a-(5-amino-2-fluorophenyl)-3,4a-difluoro-3-methylhexahydrofuro[3,4-b]pyridine-2(1H)-thione and 5-chloropicolinic acid ¹H NMR (600 MHz, DMSO) δ 10.76 (s, 1H), 8.79 (dd, J=2.4, 0.7 Hz, 1H), 8.20 (dd, J=8.4, 2.4 Hz, 1H), 8.16 (dd, J=8.4, 0.7 Hz, 1H), 7.93-7.89 (m, 2H), 7.15-7.11 (m, 1H), 6.11 (s, 2H), 4.51 (d, J=8.9 Hz, 1H), 4.17 (dd, J=17.0, 10.7 Hz, 1H), 3.91 (dd, J=25.3, 10.8 Hz, 1H), 3.86-3.81 (m, 1H), 2.59-2.51 (m, 2H), 1.69 (d, J=24.2 Hz, 3H). LC-MS (m/z) 438.83 (MH⁺); t_{R}=0.52 (Method A).

### Example 7 N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-methoxypyrazine-2-carboxamide

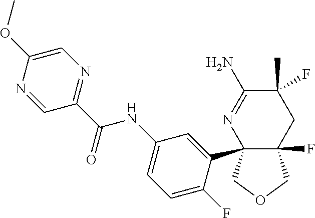 [SMILES: COc1cnc(C(=O)Nc2ccc(F)c([C@]34COC[C@@]3(F)C[C@@](C)(F)C(N)=N4)c2)cn1]

Prepared from (3R,4aR,7aS)-7a-(5-amino-2-fluorophenyl)-3,4a-difluoro-3-methylhexahydrofuro[3,4-b]pyridine-2(1H)-thione and 5-methoxypyrazine-2-carboxylic acid ¹H NMR (600 MHz, DMSO) δ 10.65 (s, 1H), 8.88 (d, J=1.3 Hz, 1H), 8.42 (d, J=1.3 Hz, 1H), 7.83 (dd, J=7.1, 2.6 Hz, 1H), 7.82-7.78 (m, 1H), 7.13 (dd, J=12.0, 8.8 Hz, 1H), 6.27 (bs, 2H), 4.65 (d, J=7.5 Hz, 1H), 4.11 (dd, J=28.0, 10.7 Hz, 1H), 4.02 (s, 3H), 4.05-3.96 (m, 1H), 3.89 (dd, J=8.4, 2.1 Hz, 1H), 2.49-2.41 (m, 1H), 2.21 (ddd, J=27.9, 14.9, 12.7 Hz, 1H), 1.66 (d, J=22.8 Hz, 3H). LC-MS (m/z) 435.4 (MH⁺); t_{R}=0.48 (Method A).

### Example 8 N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-(difluoromethyl)pyrazine-2-carboxamide

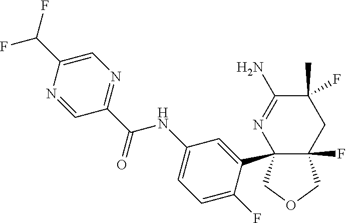 [SMILES: C[C@@]1(F)C[C@]2(F)COC[C@]2(c2cc(NC(=O)c3cnc(C(F)F)cn3)ccc2F)N=C1N]

Prepared from (3R,4aR,7aS)-7a-(5-amino-2-fluorophenyl)-3,4a-difluoro-3-methylhexahydrofuro[3,4-b]pyridine-2(1H)-thione and 5-(difluoromethyl)pyrazine-2-carboxylic acid 1H NMR (600 MHz, DMSO) δ 11.05 (s, 1H), 9.39 (d, J=1.2 Hz, 1H), 9.10 (s, 1H), 7.91-7.82 (m, 2H), 7.31 (d, J=54.0 Hz, 1H), 7.18 (dd, J=11.8, 8.8 Hz, 1H), 6.31 (bs, 2H), 4.66 (d, J=7.5 Hz, 1H), 4.12 (dd, J=27.8, 10.7 Hz, 1H), 4.02 (dd, J=25.6, 10.7 Hz, 1H), 3.91 (dd, J=8.4, 2.1 Hz, 1H), 2.51 (dt, J=3.6, 1.8 Hz, 1H), 2.21 (ddd, J=14.8, 13.9, 9.5 Hz, 1H), 1.68 (d, J=22.8 Hz, 3H). LC-MS (m/z) 455.38 (MH⁺); t_{R}=0.47 (Method A).

### Example 9 N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-4-methylthiazole-2-carboxamide

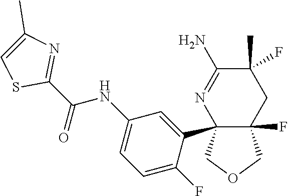 [SMILES: Cc1csc(C(=O)Nc2ccc(F)c([C@]34COC[C@@]3(F)C[C@@](C)(F)C(N)=N4)c2)n1]

Prepared from (3R,4aR,7aS)-7a-(5-amino-2-fluorophenyl)-3,4a-difluoro-3-methylhexahydrofuro[3,4-b]pyridine-2(1H)-thione and 4-methylthiazole-2-carboxylic acid ¹H NMR (600 MHz, DMSO) δ 10.88 (s, 1H), 7.83-7.77 (m, 2H), 7.70 (d, J=0.9 Hz, 1H), 7.14 (dd, J=11.9, 8.6 Hz, 1H), 6.30 (bs, 2H), 4.68-4.61 (m, 1H), 4.11 (dd, J=28.1, 10.7 Hz, 1H), 4.01 (dd, J=25.7, 10.7 Hz, 1H), 3.90 (dd, J=8.4, 2.2 Hz, 1H), 2.51 (s, 3H), 2.50-2.40 (m, 1H), 2.19 (ddd, J=27.8, 14.9, 12.7 Hz, 1H), 1.66 (d, J=22.8 Hz, 3H). LC-MS (m/z) 424.44 (MH⁺); t_{R}=0.48 (Method A).

### Example 10 N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-2-methyloxazole-4-carboxamide

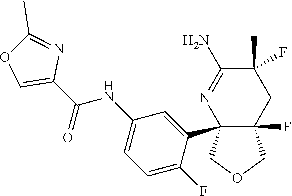 [SMILES: Cc1nc(C(=O)Nc2ccc(F)c([C@]34COC[C@@]3(F)C[C@@](C)(F)C(N)=N4)c2)co1]

Prepared from (3R,4aR,7aS)-7a-(5-amino-2-fluorophenyl)-3,4a-difluoro-3-methylhexahydrofuro[3,4-b]pyridine-2(1H)-thione and 2-methyloxazole-4-carboxylic acid ¹H NMR (600 MHz, DMSO) δ 10.29 (s, 1H), 8.65 (s, 1H), 7.78 (dd, J=7.1, 2.7 Hz, 1H), 7.72 (ddd, J=8.7, 3.9, 2.8 Hz, 1H), 7.11 (dd, J=12.0, 8.8 Hz, 1H), 6.29 (bs, 2H), 4.65 (dd, J=8.3, 1.1 Hz, 1H), 4.11 (dd, J=28.6, 10.7 Hz, 1H), 4.01 (dd, J=25.8, 10.7 Hz, 1H), 3.90 (dd, J=8.4, 2.2 Hz, 1H), 2.52 (s, 3H), 2.48-2.38 (m, 1H), 2.26-2.12 (m, 1H), 1.66 (d, J=22.8 Hz, 3H). LC-MS (m/z) 408.37 (MH⁺); t_{R}=0.41 (Method A).

### Example 11 N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-methoxypicolinamide

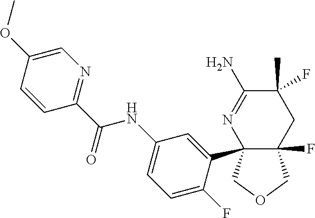 [SMILES: COc1ccc(C(=O)Nc2ccc(F)c([C@]34COC[C@@]3(F)C[C@@](C)(F)C(N)=N4)c2)nc1]

Prepared from (3R,4aR,7aS)-7a-(5-amino-2-fluorophenyl)-3,4a-difluoro-3-methylhexahydrofuro[3,4-b]pyridine-2(1H)-thione and 5-methoxypicolinic acid ¹H NMR (600 MHz, DMSO) δ 10.61 (s, 1H), 8.40 (d, J=2.8 Hz, 1H), 8.13 (d, J=8.7 Hz, 1H), 7.85 (dd, J=7.1, 2.6 Hz, 1H), 7.83-7.79 (m, 1H), 7.61 (dd, J=8.7, 2.9 Hz, 1H), 7.13 (dd, J=11.9, 8.8 Hz, 1H), 6.28 (bs, 2H), 4.66 (d, J=7.8 Hz, 1H), 4.11 (dt, J=27.0, 9.1 Hz, 1H), 4.01 (dd, J=25.8, 10.7 Hz, 1H), 3.94 (s, 3H), 3.91 (dd, J=8.3, 2.0 Hz, 1H), 2.46 (ddd, J=14.7, 10.6, 3.3 Hz, 1H), 2.23 (ddd, J=27.8, 14.8, 12.7 Hz, 1H), 1.68 (d, J=22.8 Hz, 3H). LC-MS (m/z) 434.41 (MH⁺); t_{R}=0.49 (Method A).

### Example 12 N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-fluoropicolinamide

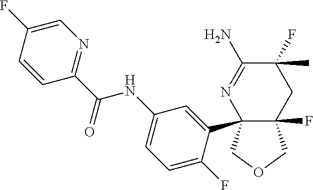 [SMILES: C[C@@]1(F)C[C@]2(F)COC[C@]2(c2cc(NC(=O)c3ccc(F)cn3)ccc2F)N=C1N]

Prepared from (3R,4aR,7aS)-7a-(5-amino-2-fluorophenyl)-3,4a-difluoro-3-methylhexahydrofuro[3,4-b]pyridine-2(1H)-thione and 5-fluoropicolinic acid 1H NMR (600 MHz, DMSO) δ 10.78 (s, 1H), 8.74 (d, J=2.8 Hz, 1H), 8.23 (dd, J=8.8, 4.6 Hz, 1H), 7.98 (td, J=8.7, 2.9 Hz, 1H), 7.85 (t, J=8.1 Hz, 2H), 7.16 (dd, J=11.8, 8.7 Hz, 1H), 6.35 (bs, 2H), 4.65 (d, J=7.6 Hz, 1H), 4.12 (dd, J=27.1, 10.7 Hz, 1H), 4.01 (dd, J=25.4, 10.7 Hz, 1H), 3.97-3.90 (m, 1H), 2.57-2.43 (m, 1H), 2.33-2.17 (m, 1H), 1.68 (d, J=22.9 Hz, 3H). LC-MS (m/z) 422.37 (MH⁺); t_{R}=0.5 (Method A).

### Example 13 N-(3-((3R,4aR,7aS)-2-amino-3,4a-difluoro-3-methyl-3,4,4a,5-tetrahydrofuro[3,4-b]pyridin-7a-yl)-4-fluorophenyl)-5-(methoxy-d₃)pyrazine-2-carboxamide

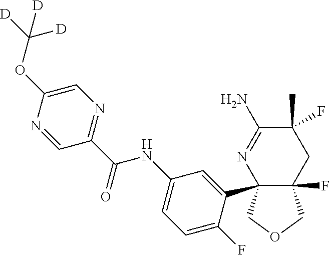 [SMILES: [2H]C([2H])([2H])Oc1cnc(C(=O)Nc2ccc(F)c([C@]34COC[C@@]3(F)C[C@@](C)(F)C(N)=N4)c2)cn1]

Prepared from (3R,4aR,7aS)-7a-(5-amino-2-fluorophenyl)-3,4a-difluoro-3-methylhexahydrofuro[3,4-b]pyridine-2(1H)-thione and 5-(methoxy-d₃)pyrazine-2-carboxylic acid ¹H NMR (600 MHz, DMSO) δ 10.65 (s, 1H), 8.88 (s, 1H), 8.41 (d, J=1.1 Hz, 1H), 7.85-7.82 (m, 1H), 7.82-7.79 (m, 1H), 7.14 (dd, J=11.9, 8.8 Hz, 1H), 6.33 (bs, 2H), 4.67-4.62 (m, 1H), 4.15-4.07 (m, 1H), 4.01 (dd, J=26.1, 9.9 Hz, 1H), 3.90 (d, J=8.2 Hz, 1H), 2.50-2.40 (m, 1H), 2.28-2.15 (m, 1H), 1.66 (d, J=22.8 Hz, 3H). LC-MS (m/z) 438.42 (MH⁺); t_{R}=0.49 (Method A).

### Example 14 N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-chloropicolinamide

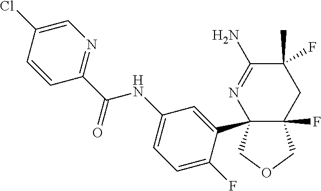 [SMILES: C[C@@]1(F)C[C@]2(F)COC[C@]2(c2cc(NC(=O)c3ccc(Cl)cn3)ccc2F)N=C1N]

Prepared from (3R,4aR,7aS)-7a-(5-amino-2-fluorophenyl)-3,4a-difluoro-3-methylhexahydrofuro[3,4-b]pyridine-2(1H)-thione and 5-chloropicolinic acid ¹H NMR (600 MHz, DMSO) δ 10.83 (s, 1H), 8.79 (d, J=1.9 Hz, 1H), 8.21-8.18 (m, 1H), 8.16 (d, J=8.3 Hz, 1H), 7.87-7.80 (m, 2H), 7.15 (dd, J=11.9, 8.6 Hz, 1H), 6.29 (bs, 2H), 4.66 (d, J=7.8 Hz, 1H), 4.12 (dd, J=28.0, 10.7 Hz, 1H), 4.01 (dd, J=25.7, 10.7 Hz, 1H), 3.91 (dd, J=8.4, 1.9 Hz, 1H), 2.50-2.42 (m, 1H), 2.22 (ddd, J=27.8, 14.8, 12.9 Hz, 1H), 1.67 (d, J=22.8 Hz, 3H). LC-MS (m/z) 438.83 (MH⁺); t_{R}=0.53 (Method A).

### Example 15 N-(3-((3R,4aS,7aS)-2-amino-3-fluoro-3-(fluoromethyl)-3,4,4a,5-tetrahydrofuro[3,4-b]pyridin-7a-yl)-4-fluorophenyl)-5-methoxypicolinamide

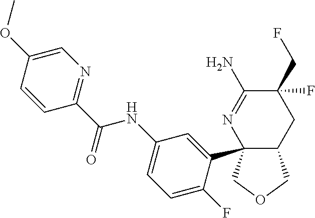 [SMILES: COc1ccc(C(=O)Nc2ccc(F)c([C@]34COC[C@H]3C[C@](F)(CF)C(N)=N4)c2)nc1]

Prepared from (3S,4aS,7aS)-7a-(5-amino-2-fluorophenyl)-3-fluoro-3-(fluoromethyl)hexahydrofuro[3,4-b]pyridine-2(1H)-thione and 5-methoxypicolinic acid ¹H NMR (600 MHz, CDCl₃) δ 9.83 (s, 1H), 8.26 (dd, J=2.8, 0.5 Hz, 1H), 8.22 (dd, J=8.7, 0.5 Hz, 1H), 7.91 (ddd, J=8.8, 4.1, 2.8 Hz, 1H), 7.57 (dd, J=7.2, 2.7 Hz, 1H), 7.33 (dd, J=8.7, 2.9 Hz, 1H), 7.08 (dd, J=11.7, 8.8 Hz, 1H), 4.68-4.47 (m, 2H), 4.34 (dd, J=9.1, 1.6 Hz, 1H), 4.19 (dd, J=8.3, 7.1 Hz, 1H), 4.00 (dd, J=9.1, 1.8 Hz, 1H), 3.94 (s, 3H), 3.88 (d, J=6.2 Hz, 1H), 2.90-2.83 (m, 1H), 2.41-2.34 (m, 1H), 2.18-2.08 (m, 1H). LC-MS (m/z) 434.412 (MH⁺); t_{R}=0.46 (Method A).

### Example 16 N-(3-((3R,4aS,7aS)-2-amino-3-fluoro-3-(fluoromethyl)-3,4,4a,5-tetrahydrofuro[3,4-b]pyridin-7a-yl)-4-fluorophenyl)-5-fluoropicolinamide

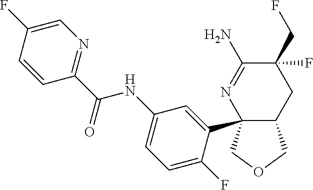 [SMILES: NC1=N[C@@]2(c3cc(NC(=O)c4ccc(F)cn4)ccc3F)COC[C@H]2C[C@]1(F)CF]

Prepared from (3S,4aS,7aS)-7a-(5-amino-2-fluorophenyl)-3-fluoro-3-(fluoromethyl)hexahydrofuro[3,4-b]pyridine-2(1H)-thione and 5-fluoropicolinic acid ¹H NMR (600 MHz, CDCl₃) δ 9.80 (s, 1H), 8.45 (d, J=2.7 Hz, 1H), 8.32 (dd, J=8.7, 4.5 Hz, 1H), 7.90 (ddd, J=8.8, 4.1, 2.8 Hz, 1H), 7.59 (ddd, J=9.2, 7.4, 2.7 Hz, 2H), 7.10 (dd, J=11.7, 8.8 Hz, 1H), 4.68-4.48 (m, 2H), 4.35 (dd, J=9.1, 1.5 Hz, 1H), 4.19 (dd, J=8.3, 7.2 Hz, 1H), 3.99 (dd, J=9.1, 1.7 Hz, 1H), 3.88 (d, J=7.6 Hz, 1H), 2.90-2.82 (m, 1H), 2.41-2.34 (m, 1H), 2.13 (ddd, J=26.5, 13.3, 2.1 Hz, 1H). LC-MS (m/z) 422.376 (MH⁺); t_{R}=0.45 (Method A).

### Example 17 N-[3-[(3R,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyridine-2-carboxamide

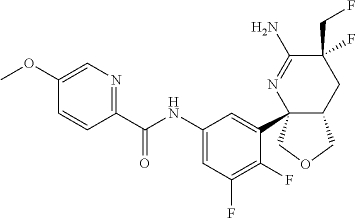 [SMILES: COc1ccc(C(=O)Nc2cc(F)c(F)c([C@]34COC[C@H]3C[C@](F)(CF)C(N)=N4)c2)nc1]

Prepared from (3S,4aS,7aS)-7a-(5-amino-2,3-difluorophenyl)-3-fluoro-3-(fluoromethyl)hexahydrofuro[3,4-b]pyridine-2(1H)-thione and 5-methoxypicolinic acid ¹H NMR (400 MHz, CDCl₃) δ 9.87 (s, 1H), 8.27-8.21 (m, 2H), 8.04-8.00 (m, 1H), 7.37 (dd, J=8.8, 2.4 Hz, 1H), 7.25-7.23 (m, 1H), 4.68-4.45 (m, 2H), 4.33 (d, J=9.6 Hz, 1H), 4.20 (t, J=8.0 Hz, 1H), 3.99 (d, J=9.2 Hz, 1H), 3.95 (s, 3H), 3.89 (d, J=8.4 Hz, 1H), 2.85 (d, J=6.4 Hz, 1H), 2.43-2.35 (m, 1H), 2.13 (q, J=13.2 Hz, 1H) LC-MS (m/z) 453.1 (MH⁺); t_{R}=1.95 (Method C).

### Example 18 N-[3-[(3R,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyrazine-2-carboxamide

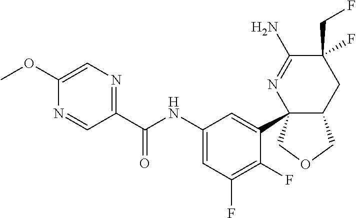 [SMILES: COc1cnc(C(=O)Nc2cc(F)c(F)c([C@]34COC[C@H]3C[C@](F)(CF)C(N)=N4)c2)cn1]

Prepared from (3S,4aS,7aS)-7a-(5-amino-2,3-difluorophenyl)-3-fluoro-3-(fluoromethyl)hexahydrofuro[3,4-b]pyridine-2(1H)-thione and 5-methoxypyrazine-2-carboxylic acid ¹H NMR (400 MHz, CDCl₃) δ 9.53 (s, 1H), 9.01 (s, 1H), 8.16 (s, 1H), 8.03-7.98 (m, 1H), 7.23-7.22 (m, 1H), 4.70-4.46 (m, 2H), 4.32 (d, J=9.2 Hz, 1H), 4.19 (t, J=7.8 Hz, 1H), 4.08 (s, 3H), 3.98 (d, J=9.6 Hz, 1H), 3.90 (d, J=8.0 Hz, 1H), 2.84 (d, J=6.4 Hz, 1H), 2.42-2.35 (m, 1H), 2.14 (q, J=13.2 Hz, 1H) LC-MS (m/z) 454.1 (MH⁺); t_{R}=1.92 (Method C).

### Example 19 N-[3-[(3R,4aS,7aS)-2-amino-3-fluoro-3-(fluoromethyl)-4,4a,5,7-tetrahydrofuro[3,4-b]pyridin-7a-yl]-4,5-difluoro-phenyl]-5-fluoro-pyridine-2-carboxamide

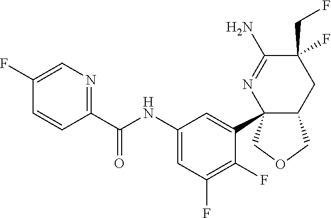 [SMILES: NC1=N[C@@]2(c3cc(NC(=O)c4ccc(F)cn4)cc(F)c3F)COC[C@H]2C[C@]1(F)CF]

Prepared from (3S,4aS,7aS)-7a-(5-amino-2,3-difluorophenyl)-3-fluoro-3-(fluoromethyl)hexahydrofuro[3,4-b]pyridine-2(1H)-thione and 5-fluoropicolinic acid ¹H NMR (400 MHz, CDCl₃) δ 9.87 (s, 1H), 8.46 (d, J=2 Hz 1H), 8.33-8.30 (m, 1H), 8.04-8.02 (m, 1H), 7.61 (td, J=8.4, 2.4 Hz, 1 Hz), 7.25 (s, 1H), 4.67-4.55 (m, 2H), 4.31 (d, J=9.2 Hz, 1H), 4.21 (t, J=7.8 Hz, 1H), 4.03 (d, J=9.2 Hz, 1H), 3.90 (d, J=8.4 Hz, 1H), 2.87 (d, J=6.8 Hz, 1H), 2.44-2.37 (m, 1H), 2.22-2.10 (m, 1H) LC-MS (m/z) 444.1 (MH⁺); t_{R}=1.93 (Method C).

### Example 20 N-(3-((3S,4aS,7aS)-2-amino-3-fluoro-3-(fluoromethyl)-3,4,4a,5-tetrahydrofuro[3,4-b]pyridin-7a-yl)-4-fluorophenyl)-5-fluoropicolinamide

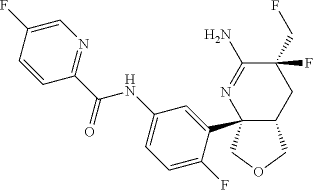 [SMILES: NC1=N[C@@]2(c3cc(NC(=O)c4ccc(F)cn4)ccc3F)COC[C@H]2C[C@@]1(F)CF]

Prepared from (3R,4aS,7aS)-7a-(5-amino-2-fluorophenyl)-3-fluoro-3-(fluoromethyl)hexahydrofuro[3,4-b]pyridine-2(1H)-thione and 5-fluoropicolinic acid ¹H NMR (600 MHz, DMSO) δ 10.68 (s, 1H), 8.74 (d, J=2.8 Hz, 1H), 8.23 (dd, J=8.8, 4.6 Hz, 1H), 8.03-7.96 (m, 2H), 7.91-7.86 (m, 1H), 7.16 (dd, J=11.3, 9.0 Hz, 1H), 6.08 (bs, 2H), 4.94 (ddd, J=48.8, 17.2, 11.2 Hz, 1H), 4.73 (ddd, J=45.8, 34.6, 11.1 Hz, 1H), 4.04 (d, J=8.7 Hz, 1H), 3.93 (t, J=8.6 Hz, 1H), 3.81 (d, J=7.2 Hz, 1H), 3.72 (d, J=8.4 Hz, 1H), 2.85 (q, J=8.7 Hz, 1H), 2.36-2.25 (m, 1H), 2.17-2.06 (m, 1H). LC-MS (m/z) 422.376 (MH⁺); t_{R}=0.48 (Method B).

### Example 21 N-(3-((3S,4aS,7aS)-2-amino-3-fluoro-3-(fluoromethyl)-3,4,4a,5,7,7a-hexahydrofuro[3,4-b]pyridin-7a-yl)-4-fluorophenyl)-5-methoxypicolinamide

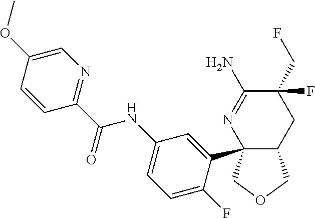 [SMILES: COc1ccc(C(=O)Nc2ccc(F)c([C@]34COC[C@H]3C[C@@](F)(CF)C(N)=N4)c2)nc1]

Prepared from (3R,4aS,7aS)-7a-(5-amino-2-fluorophenyl)-3-fluoro-3-(fluoromethyl)hexahydrofuro[3,4-b]pyridine-2(1H)-thione and 5-methoxypicolinic acid ¹H NMR (600 MHz, DMSO) δ 10.53 (s, 1H), 8.39 (d, J=2.9 Hz, 1H), 8.13 (d, J=8.7 Hz, 1H), 7.98 (dd, J=7.3, 2.6 Hz, 1H), 7.89 (ddd, J=8.7, 4.0, 2.8 Hz, 1H), 7.62 (dd, J=8.8, 2.9 Hz, 1H), 7.14 (dd, J=11.5, 8.9 Hz, 1H), 6.08 (bs, 2H), 4.94 (ddd, J=48.8, 17.3, 11.1 Hz, 1H), 4.72 (ddd, J=46.0, 34.6, 11.1 Hz, 1H), 4.06-4.01 (m, 1H), 3.96-3.90 (m, 1H), 3.93 (s), 3.84-3.78 (m, 1H), 3.74-3.69 (m, 1H), 2.88-2.82 (m, 1H), 2.37-2.25 (m, 1H), 2.17-2.07 (m, 1H). LC-MS (m/z) 434.41 (MH⁺); t_{R}=0.49 (Method B).

### Example 22 N-(3-((3S,4aS,7aS)-2-amino-3-fluoro-3-(fluoromethyl)-3,4,4a,5-tetrahydrofuro[3,4-b]pyridin-7a-yl)-4-fluorophenyl)-5-(methoxy-d₃)picolinamide

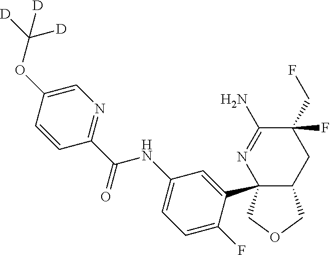 [SMILES: [2H]C([2H])([2H])Oc1ccc(C(=O)Nc2ccc(F)c([C@]34COC[C@H]3C[C@@](F)(CF)C(N)=N4)c2)nc1]

Prepared from (3R,4aS,7aS)-7a-(5-amino-2-fluorophenyl)-3-fluoro-3-(fluoromethyl)hexahydrofuro[3,4-b]pyridine-2(1H)-thione and 5-(methoxy-d₃)picolinic acid ¹H NMR (600 MHz, DMSO) δ 10.56 (s, 1H), 8.41-8.38 (m, 1H), 8.14-8.11 (m, 1H), 7.90-7.85 (m, 1H), 7.76 (dd, J=7.3, 2.4 Hz, 1H), 7.61 (dd, J=8.7, 2.9 Hz, 1H), 7.15 (dd, J=11.9, 8.8 Hz, 1H), 6.25 (s, 2H), 4.83 (ddd, J=48.5, 16.7, 11.1 Hz, 1H), 4.57 (ddd, J=46.2, 31.6, 11.0 Hz, 1H), 4.23 (d, J=8.6 Hz, 1H), 3.97 (t, J=8.0 Hz, 1H), 3.85-3.80 (m, 1H), 3.75-3.69 (m, 1H), 2.83-2.76 (m, 1H), 2.19-2.01 (m, 2H) LC-MS (m/z) 438.1 (MH⁺); t_{R}=0.49 (Method B).

### Example 23 N-[3-[(3S,4aS,7aS)-2-amino-3-fluoro-3-(fluoromethyl)-4,4a,5,7-tetrahydrofuro[3,4-b]pyridin-7a-yl]-4,5-difluoro-phenyl]-5-fluoro-pyridine-2-carboxamide

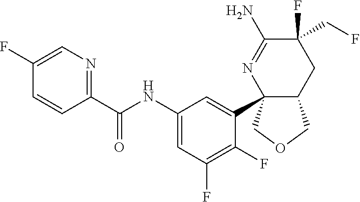 [SMILES: NC1=N[C@@]2(c3cc(NC(=O)c4ccc(F)cn4)cc(F)c3F)COC[C@H]2C[C@@]1(F)CF]

Prepared from (3R,4aS,7aS)-7a-(5-amino-2,3-difluorophenyl)-3-fluoro-3-(fluoromethyl)hexahydrofuro[3,4-b]pyridine-2(1H)-thione and 5-fluoropicolinic acid ¹H NMR (400 MHz, CDCl₃) δ 9.86 (s, 1H), 8.47 (s, 1H), 8.35-8.32 (m, 1H), 8.03 (t, J=8.6 Hz, 1H), 7.63 (t, J=8.2 Hz, 1H), 7.44 (s, 1H), 5.06-4.76 (m, 2H), 4.23-4.15 (m, 2H), 3.91 (t, J=3.2 Hz, 2H), 3.05 (s, 1H), 2.46 (d, J=6.8 Hz, 1H), 2.08-1.98 (m, 1H) LC-MS (m/z) 444.1 (MH⁺); t_{R}=2.26 (Method D).

### Example 24 N-[3-[(3S,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyrazine-2-carboxamide

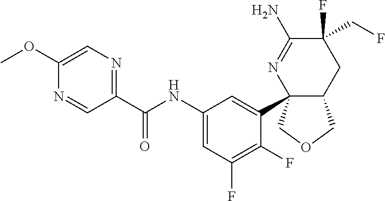 [SMILES: COc1cnc(C(=O)Nc2cc(F)c(F)c([C@]34COC[C@H]3C[C@@](F)(CF)C(N)=N4)c2)cn1]

Prepared from (3R,4aS,7aS)-7a-(5-amino-2,3-difluorophenyl)-3-fluoro-3-(fluoromethyl)hexahydrofuro[3,4-b]pyridine-2(1H)-thione and 5-methoxypyrazine-2-carboxylic acid ¹H NMR (400 MHz, CDCl₃) (δ 9.54 (s, 1H), 9.02 (s, 1H), 8.16 (s, 1H), 8.02 (t, J=8.2 Hz, 1H), 7.42 (s, 1H), 5.05-4.82 (m, 2H), 4.22-4.14 (m, 2H), 4.08 (s, 1H), 3.91 (t, J=9.6 Hz, 2H), 3.04 (s, 1H), 2.45 (d, J=14.0 Hz, 1H), 2.07-2.02 (m, 1H) LC-MS (m/z) 454.1 (MH⁺); t_{R}=2.00 (Method C).

### Example 25 N-[3-[(3S,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyridine-2-carboxamide

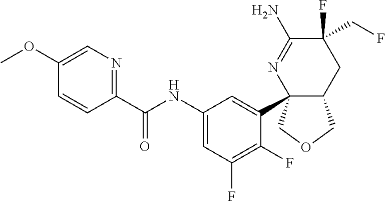 [SMILES: COc1ccc(C(=O)Nc2cc(F)c(F)c([C@]34COC[C@H]3C[C@@](F)(CF)C(N)=N4)c2)nc1]

Prepared from (3R,4aS,7aS)-7a-(5-amino-2,3-difluorophenyl)-3-fluoro-3-(fluoromethyl)hexahydrofuro[3,4-b]pyridine-2(1H)-thione and 5-methoxypicolinic acid ¹H NMR (400 MHz, CDCl₃) (δ 9.88 (s, 1H), 8.27-8.23 (m, 2H), 8.03 (t, J=9.6 Hz, 1H), 7.42 (s, 1H), 7.35 (d, J=8.8 Hz, 1H), 5.06-4.06 (m, 2H), 4.22-4.14 (m, 2H), 3.95 (s, 3H), 3.93-3.91 (m, 2H), 3.04 (s, 1H), 2.46 (d, J=13.6 Hz, 1H), 2.08-2.00 (m, 1H) LC-MS (m/z) 453.1 (MH⁺); t_{R}=2.04 (Method C)

### Pharmacological Testing

### BACE1 Binding Assay

The binding assay was performed as SPA-based assay using a biotinylated form of human BACE1 recombinantly expressed and subsequently purified from Freestyle HEK293 cells. The binding assay was run in a 50 mM sodium acetate buffer, pH 4.5 containing 50 mM NaCl and 0.03% Tween-20 in white clear bottom 384 plates (Corning #3653). 10 nM (final concentration) radioligand ([³H]—N-((1S,2R)-1-benzyl-3-cyclopropylamino-2-hydroxy-propyl)-5-(methanesulfonyl-methyl-amino)-N—((R)-1-phenyl-ethyl)-isophthalamide) (TRQ11569 purchased from GE Healthcare) was mixed with test compound at a given concentration, 6 nM (final concentration) human BACE1 and 25 μg Streptavidin coated PVT core SPA beads (RPNQ0007, GE Healthcare Life Sciences) in a total volume of 40 μl. Several concentrations of each test compound were tested in the assay for 10₅₀ determination. The plates were incubated for one hour at room temperature and counted in a Wallac Trilux counter. Total and non-specific binding were determined using buffer and 1 μM (final concentration) of the high affinity BACE1 reference inhibitor (S)-6-[3-chloro-5-(5-prop-1-ynyl-pyridin-3-yl)-thiophen-2-yl]-2-imino-3,6-dimethyl-tetrahydro-pyrimidin-4-one, respectively. For each test compound, a 10₅₀ value (the concentration mediating 50% inhibition of the specific binding of the radioligand) was determined from concentration-response curve and used to calculate the Kᵢ from the equation Kᵢ=IC₅₀/(1+L/K_{d}), where L and K_{d} are the final concentration of the radioligand used in the assay and the dissociation constant of the radioligand, respectively. The K_{d} of the radioligand was determined from saturation binding experiments.

TABLE 1
binding affinity of selected compounds

| Compound | BACE1 |
|---|---|
| No | Ki (nM) |
| 1 | 24 |
| 2 | 31 |
| 3 | 48 |
| 4 | 27 |
| 5 | 17 |
| 6 | 6.4 |
| 7 | 11 |
| 8 | 14 |
| 9 | 23 |
| 10 | 22 |
| 11 | 16 |
| 12 | 24 |
| 13 | 29 |
| 14 | 9.3 |
| 15 | 65 |
| 16 | 88 |
| 17 | 99 |
| 18 | 110 |
| 19 | 340 |
| 20 | 140 |
| 21 | 55 |
| 22 | 51 |
| 23 | 200 |
| 24 | 130 |
| 25 | 230 |

### BACE1 Efficacy Assay

The efficacy assay was performed as a FRET-based assay using a commercially available BACE1 kit (Life Technologies, P2985). 2 μl test compound at 10 μM (final concentration) and 15 μl BACE1 enzyme from the kit (final concentration 3 nM) were preincubated for 15 minutes at room temperature before addition of 15 μl of substrate from the kit (250 nM final concentration) and incubated for additional 90 minutes at room temperature. The assay plate was subsequently read in a Pherastar (Ex540/Em590). The enzyme activity observed in presence of test compound were normalized to the enzyme activity observed in presence of buffer and 10 μM (final concentration) of the high affinity BACE1 reference inhibitor (S)-6-[3-Chloro-5-(5-prop-1-ynyl-pyridin-3-yl)-thiophen-2-yl]-2-imino-3,6-dimethyl-tetrahydropyrimidin-4-one, respectively. The efficacy of the test compounds was evaluated at 10 μM (final concentration) and defined as the percent inhibition of the enzyme activity using the equation % inhibition=100%−normalized enzyme activity in percent.

TABLE 2
BACE1 activity of selected compounds

| Compound | BACE1 inhibition |
|---|---|
| No | at 10 μM (%) |
| 7 | 104 |
| 12 | 103 |
| 15 | 111 |
| 16 | 110 |
| 17 | 105 |
| 18 | 105 |
| 20 | 103 |
| 21 | 103 |
| 22 | 99 |
| 23 | 103 |
| 24 | 103 |

Assessment of Aβ Peptide Levels in Rat Brain and Plasma Following BACE1 Inhibition. Animals.

All rat care and experimental procedures were approved by Lundbeck Veterinary Staff, according to Danish legislature. The rats were maintained in a barrier facility with a 12/12-h light/dark cycle and ad libitum food and water access.

### Treatment of Naïve Rats.

Young adult Male Sprague Dawley rats of approximately 250 g weight were purchased from Charles River and received 0-30 mg/kg of vehicle (10% HP betaCD+1M MeSO₄, pH 2.5) or test compounds (dissolved in vehicle) only by oral gavage (p.o). The compounds are dosed at a volume of 5 ml/kg. Cohorts of 5-10 animals were established for each treatment condition.

The animals undergoing treatment were closely monitored by veterinary staff for any signs of toxicity. Monitoring parameters included body weight, physical appearance, changes in coat appearance, occurrence of unprovoked behavior, and blunted or exaggerated responses to external stimuli.

### Tissue Collection.

At T=180 minutes after initial dosing the animals were stunned and decapitated with a guillotine. Trunk-blood was sampled in EDTA coated tubes after decapitation of the animal. The blood was centrifuged at 2200G at 4° C. for 15 minutes and the plasma was collected and frozen at −80° C. The blood was aliquoted for Aβ ELISA and DMPK analysis. Immediately following sacrifice, the brain was extracted and split into 2 halves. The right hemibrains were snap frozen on dry ice and stored at −80° C. The left half was dissected; with the front forebrain taken for Aβ ELISA and the remainder used for DMPK analysis. These samples were also snap frozen on dry ice and stored at −80° C. until use for analysis.

### Tissue Processing.

The cortex samples were thawed slightly on wet ice before they were homogenized with a small volume dispersing instrument (T10 basic ULTRA-TURRAX®) which was set at speed 5 for approximately 5-7 sec. The tissue was processed in a 10 times volume of the weight, for example 100 mg of tissue was homogenized in 1000 μL of Homogenization buffer. Homogenization buffer: 50 ml Milli Q water+50 nM NaCl+0.2% Diethylamin (DEA)+1 tablet of Complete Protease inhibitor cocktail+1 nM 4-(2-aminoethyl) benzenesulfonyl fluoride hydrochloride irreversible serine protease inhibitor (AEBSF).

After homogenization 450 μL aliquots of the samples are collected into a 1.5 ml Eppendorf tube and placed on wet ice, 0.5% NP-40 (50 ul) was added to all samples and then they were incubated on ice for 30 min. After which all samples were sonicated using an Ultrasonic homogenizer with 20 kHz homogeneous sound (SONOPLUS HD2070, Bandelin Electronic) 10 pulse set at 12-13% power to extract all the Aβ species. The samples were then centrifuged (Ole Dich 157 MPRF Micro centrifuge) at 20000G for 20 minutes at 4° C. After centrifugation 285 μL of the supernatant was pipetted into 600 μL microtubes tubes and neutralized with 15 μL of 1M Tris-HCL buffer.

### ELISA Protocol.

WAKO 294-62501 Human/Rat Abeta amyloid (40) kit was used for all ELISA analyses. 30 μL plasma samples or 30 μL of the cortex supernatants generated as described above were placed in 600 μL microtubes tubes on wet ice. To this 30 μL of 8M urea (AppliChem A1049, 9025) was added to generate a 2-fold dilution. Both plasma and cortex supernatants were incubated on ice for 30 min. Standard rows were prepared from the standard peptide stock provided in the kit and standard diluent containing 1.6M urea (200 μL 8M urea+800 μL of standard diluent) and 0.8M urea (400 μL 8M Urea+3600 μL Standard diluent). A serial 2-fold dilution of Aβ40 from 100 pmol/ml to 0 pmol/L was prepared for the assay.

After incubation with urea, all samples were further diluted by addition of 5 times standard diluent from the Kit. This was done by adding 240 μL Standard Diluent to 60 μL sample/urea mixture, which was then mixed well. 100 μL of each diluted sample was pipetted into designated wells of the ELISA plate in duplicates. The plate was then covered and incubated overnight at 4° C. The following day, the ELISA kit was brought to room temperature before use. The incubated plate was washed 5 times with the 20× washing solution diluted in Milli Q water. 100 μL HRP-conjugate was applied to each well, and the plate was covered and incubates at 4° C. for 1 hr. The wash was repeated again for 5 times. 100 μL 3,3′,5,5′-Tetramethylbenzidine (TMB) solution was applied to each well and the plate was covered and incubated in the dark at room temperature for 30 minutes. 100 μL STOP-solution was next applied to each well, and the plate was read at 450 nm wavelength in a spectrophotometer (Labsystems Multiscan Ascent) within 30 min of adding the STOP-solution to the wells.

Concentration of Aβ in the samples was determined based on a standard curve generated from standards containing known concentrations of synthetic Aβ40. Those skilled in the art will appreciate that diethylamine (DEA) and urea extractions will release soluble Aβ, and insoluble Aβ respectively. Since the ELISA kit is validated and widely used, it is accepted that as long as the treatment conditions and assay conditions are the same for each compound tested, then the assay should yield consistent robust data for the compounds tested and produce minimal discrepancies.

### Data Analysis

To determine the concentration of Aβ40 peptide in the samples, the interpolated values of the samples loaded on plates are multiplied by 20 to account for the dilutions made when the volumes of DEA, urea and neutralization solution were added up. Values are calculated as percentage change in Aβ40 peptide compared to vehicle treated animals.

The results of the administration of representative Compounds of Examples 1, 2, 5, 7 and 22 at doses of 10 mg/kg and 10 mg/kg p.o., of brain and plasma samples collected at 3 hours post dose and then following exposures, as described above, are tabulated below.

TABLE 3
Results for Example 1

| | | | Brain/Plasma | Aβ40 |
|---|---|---|---|---|
| | Dose (mg/kg) | Exp (ng/g) | ratio | reduction (%) |
| Brain Rat | 10 | 194 | 1.06 | 34 |
| Plasma Rat | | 184 | | 38 |
| Brain Rat | 30 | 951 | 1.50 | 54 |
| Plasma Rat | | 634 | | 52 |

TABLE 4
Results for Example 2

| | | | Brain/Plasma | Aβ40 |
|---|---|---|---|---|
| | Dose (mg/kg) | Exp (ng/g) | ratio | reduction (%) |
| Brain Rat | 10 | 37 | 0.54 | 25 |
| Plasma Rat | | 69 | | 48 |
| Brain Rat | 30 | 136 | 0.56 | 24 |
| Plasma Rat | | 244 | | 47 |

TABLE 5
Results for Example 5

| | | | Brain/Plasma | Aβ40 |
|---|---|---|---|---|
| | Dose (mg/kg) | Exp (ng/g) | ratio | reduction (%) |
| Brain Rat | 10 | 204 | 1.17 | 40 |
| Plasma Rat | | 174 | | 41 |
| Brain Rat | 30 | 695 | 1.16 | 47 |
| Plasma Rat | | 598 | | 45 |

TABLE 6
Results for Example 7

| | | | Brain/Plasma | Aβ40 |
|---|---|---|---|---|
| | Dose (mg/kg) | Exp (ng/g) | ratio | reduction (%) |
| Brain Rat | 10 | 93 | 1.05 | 48 |
| Plasma Rat | | 88 | | 42 |
| Brain Rat | 30 | 479 | 2.01 | 64 |
| Plasma Rat | | 239 | | 50 |

TABLE 7
Results for Example 22

| | | | Brain/Plasma | Aβ40 |
|---|---|---|---|---|
| | Dose (mg/kg) | Exp (ng/g) | ratio | reduction (%) |
| Brain Rat | 10 | 153 | 0.38 | 23 |
| Plasma Rat | | 399 | | 33 |
| Brain Rat | 30 | 776 | 0.42 | 48 |
| Plasma Rat | | 1834 | | 43 |

As shown in Tables 3 through 7, compounds of the present invention are able to penetrate the blood brain barrier and show efficacy in the CNS.

### MDCK-MDR1 Assay

The permeability of the test compounds was assessed in MDCK-MDR1 cells that were cultured to confluency (4-6 days) in a 96 transwell plate. Test compounds were diluted with the transport buffer (HBSS+1% BSA) to a concentration of 0.5 μM and applied to the apical or basolateral side of the cell monolayer. Permeation of the test compounds from A to B direction or B to A direction was determined in triplicate over a 60-minute incubation time at 37° C. and 5% CO2 with a relative humidity of 95%. Test compounds were quantified by LC-MS/MS analysis based on the peaks area ratios of analyte/IS in both the receiver and donor wells of the transwell plate. The apparent permeability coefficient Papp (cm/s) was calculated using the equation:

Papp=(dCr/dt)×Vr/(A×C0)

Where dCr/dt is the cumulative concentration of compound in the receiver chamber as a function of time (μM/s); Vr is the solution volume in the receiver chamber (0.05 mL on the apical side; 0.25 mL on the basolateral side); A is the surface area for the transport, i.e. 0.0804 cm² for the area of the monolayer; CO is the initial concentration in the donor chamber (μM).

Compounds are classified Pgp substrates when efflux ratio (Papp BA/Papp AB) is 2.

TABLE 7
Efflux ratio of selected compounds

| | MDCK-MDR1 |
|---|---|
| Compound | efflux ratio |
| 1 | 0.67 |
| 2 | 1.27 |
| 3 | 0.96 |
| 4 | 0.81 |
| 5 | 0.91 |
| 6 | 1.01 |
| 7 | 0.94 |
| 8 | 1.23 |
| 9 | 1.33 |
| 10 | 2.46 |
| 11 | 1.08 |
| 12 | 1.67 |
| 14 | 0.91 |
| 15 | 1.19 |
| 16 | 0.97 |
| 17 | 1.25 |
| 18 | 1.31 |
| 20 | 0.71 |
| 21 | 0.61 |
| 22 | 1.85 |
| 23 | 0.89 |
| 24 | 0.82 |

As shown in Table 7, the majority of the exemplified compounds of the present invention have MDCK-MDR1 efflux ratios below 2 and are thus likely to be able to cross the blood brain barrier (E Kerns, L Di, Drug-like Properties: Concepts, Structure Design and Methods (2008) Elsevier).

1. A compound of Formula I 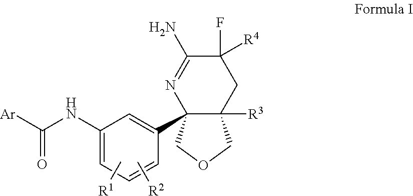
or a pharmaceutically acceptable salt thereof, wherein:
Ar is selected from the group consisting of phenyl, pyridyl, pyrimidyl, pyrazinyl, imidazolyl, pyrazolyl, thiazolyl, oxazoly and isoxazolyl, and wherein Ar is optionally substituted with one or more substituents selected from the group consisting of halogen, CN, C₁-C₆ alkyl, C₂-C₆ alkenyl, C₂-C₆ alkynyl, C₁-C₆ fluoroalkyl and C₁-C₆ alkoxy;
R¹ is selected from the group consisting of hydrogen, halogen, C₁-C₃ alkyl and C₁-C₃ fluoroalkyl;
R² is selected from the group consisting of hydrogen, halogen, C₁-C₃ alkyl and C₁-C₃ fluoroalkyl;
R³ is hydrogen or halogen;
R⁴ is C₁-C₃ alkyl or C₁-C₃ fluoroalkyl.

2. The compound according to claim 1, wherein said compound is of Formula Ia 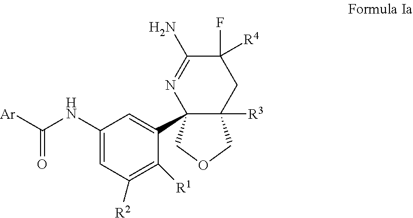
or a pharmaceutically acceptable salt thereof.

3. The compound, or a pharmaceutically acceptable salt thereof, according to claim 1, wherein R¹ is F and R² is hydrogen.

4. The compound, or a pharmaceutically acceptable salt thereof, according to claim 1, wherein R¹ and R² are F.

5. The compound, or a pharmaceutically acceptable salt thereof, according to claim 1, wherein R³ is hydrogen or F.

6. The compound, or a pharmaceutically acceptable salt thereof, according to claim 1, wherein R⁴ is methyl or fluoromethyl.

7. The compound, or a pharmaceutically acceptable salt thereof, according to claim 1, wherein R³ is hydrogen and R⁴ is C₁-C₃ fluoroalkyl.

8. The compound, or a pharmaceutically acceptable salt thereof, according to claim 1, wherein Ar is substituted with a substituent selected from the group consisting of F, Cl, C₁-C₃ alkoxy and C₁-C₃ fluoroalkyl.

9. The compound, or a pharmaceutically acceptable salt thereof, according to claim 1, wherein Ar is pyridyl.

10. The compound, or a pharmaceutically acceptable salt thereof, according to claim 1, wherein Ar is pyrazinyl.

11. The compound, or a pharmaceutically acceptable salt thereof, according to claim 1, wherein Ar is thiazolyl.

12. The compound, or a pharmaceutically acceptable salt thereof, according to claim 1, wherein Ar is oxazolyl.

13. A compound selected form the group consisting of
N-(3-((3S,4aR,7aS)-2-amino-3,4a-difluoro-3-methyl-3,4,4a,5-tetrahydrofuro[3,4-b]pyridin-7a-yl)-4-fluorophenyl)-5-(methoxy-d₃)picolinamide,
N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-fluoropicolinamide,
N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-(difluoromethyl)pyrazine-2-carboxamide,
N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-methoxypicolinamide,
N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-methoxypyrazine-2-carboxamide,
N-(3-((3S,4aR,7aS)-2-amino-3,4a-difluoro-3-methyl-3,4,4a,5,7,7a-hexahydrofuro[3,4-b]pyridin-7a-yl)-4-fluorophenyl)-5-chloropicolinamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-methoxypyrazine-2-carboxamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-(difluoromethyl)pyrazine-2-carboxamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-4-methylthiazole-2-carboxamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-2-methyloxazole-4-carboxamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-methoxypicolinamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-fluoropicolinamide,
N-(3-((3R,4aR,7aS)-2-amino-3,4a-difluoro-3-methyl-3,4,4a,5-tetrahydrofuro[3,4-b]pyridin-7a-yl)-4-fluorophenyl)-5-(methoxy-d₃)pyrazine-2-carboxamide,
N-(3-((3R,4aR,7aS)-2-amino-3,4a-difluoro-3-methyl-3,4,4a,5,7,7a-hexahydrofuro[3,4-b]pyridin-7a-yl)-4-fluorophenyl)-5-chloropicolinamide,
N-(3-((3R,4aS,7aS)-2-amino-3-fluoro-3-(fluoromethyl)-3,4,4a,5-tetrahydrofuro[3,4-b]pyridin-7a-yl)-4-fluorophenyl)-5-methoxypicolinamide,
N-(3-((3R,4aS,7aS)-2-amino-3-fluoro-3-(fluoromethyl)-3,4,4a,5-tetrahydrofuro[3,4-b]pyridin-7a-yl)-4-fluorophenyl)-5-fluoropicolinamide,
N-[3-[(3R,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyridine-2-carboxamide,
N-[3-[(3R,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyrazine-2-carboxamide,
N-[3-[(3R,4aS,7aS)-2-amino-3-fluoro-3-(fluoromethyl)-4,4a,5,7-tetrahydrofuro[3,4-b]pyridin-7a-yl]-4,5-difluoro-phenyl]-5-fluoro-pyridine-2-carboxamide,
N-(3-((3S,4aS,7aS)-2-amino-3-fluoro-3-(fluoromethyl)-3,4,4a,5-tetrahydrofuro[3,4-b]pyridin-7a-yl)-4-fluorophenyl)-5-fluoropicolinamide,
N-(3-((3S,4aS,7aS)-2-amino-3-fluoro-3-(fluoromethyl)-3,4,4a,5,7,7a-hexahydrofuro[3,4-b]pyridin-7a-yl)-4-fluorophenyl)-5-methoxypicolinamide,
N-(3-((3S,4aS,7aS)-2-amino-3-fluoro-3-(fluoromethyl)-3,4,4a,5-tetrahydrofuro[3,4-b]pyridin-7a-yl)-4-fluorophenyl)-5-(methoxy-d₃)picolinamide,
N-[3-[(3S,4aS,7aS)-2-amino-3-fluoro-3-(fluoromethyl)-4,4a,5,7-tetrahydrofuro[3,4-b]pyridin-7a-yl]-4,5-difluoro-phenyl]-5-fluoro-pyridine-2-carboxamide,
N-[3-[(3S,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyrazine-2-carboxamide and
N-[3-[(3S,4aS,7aS)-2-amino-3-fluoro-3-(fluoromethyl)-4,4a,5,7-tetrahydrofuro[3,4-b]pyridin-7a-yl]-4,5-difluoro-phenyl]-5-methoxy-pyridine-2-carboxamide,
or a pharmaceutically acceptable salt thereof.

14. (canceled)

15. A pharmaceutical composition comprising a compound, or a pharmaceutically acceptable salt thereof, according to claim 1 and a pharmaceutically acceptable carrier.

16. (canceled)

17. (canceled)

18. A method for the treatment of a disease selected from the group consisting of Alzheimer's disease, preclinical Alzheimer's disease, prodromal Alzheimer's disease, mild cognitive impairment, Down's syndrome and cerebral amyloid angiopathy, comprising administering a therapeutically effective amount of a compound, or a pharmaceutically acceptable salt thereof, according to claim 1 to a patient in need thereof.

19. A method for the treatment of a neurodegenerative or cognitive disorder, comprising administering a therapeutically effective amount of a compound, or a pharmaceutically acceptable salt thereof, according to claim 1 to a patient in need thereof.

20. A pharmaceutical composition comprising a compound, or a pharmaceutically acceptable salt thereof, according to claim 13 and a pharmaceutically acceptable carrier.

21. A method for the treatment of a disease selected from the group consisting of Alzheimer's disease, preclinical Alzheimer's disease, prodromal Alzheimer's disease, mild cognitive impairment, Down's syndrome and cerebral amyloid angiopathy, comprising administering a therapeutically effective amount of a compound, or a pharmaceutically acceptable salt thereof, according to claim 13 to a patient in need thereof.

22. A method for the treatment of a neurodegenerative or cognitive disorder, comprising administering a therapeutically effective amount of a compound, or a pharmaceutically acceptable salt thereof, according to claim 13 to a patient in need thereof.
